(19) United States
(12) Patent Application Publication
(10) Pub. No.: US 2021/0346464 A1
(43) Pub. Date: Nov. 11, 2021

(54) TREATMENT OF ANEMIA DUE TO VERY LOW, LOW, OR INTERMEDIATE RISK MYELODYSPLASTIC SYNDROMES IN SUBJECTS WITH RING SIDEROBLASTS USING ACTIVIN-ACTRll LIGAND TRAPS

(71) Applicants: Celgene Corporation, Summit, NJ (US); Acceleron Pharma Inc., Cambridge, MA (US)

(72) Inventors: Abderrahmane Laadem, Belle Mead, NJ (US); Xianjuan Zhang, Short Hills, NJ (US); Kenneth M. Attie, Boston, MA (US); Peter G. Linde, Malden, MA (US)

(21) Appl. No.: 17/289,420

(22) PCT Filed: Oct. 30, 2019
(86) PCT No.: PCT/US2019/058803

§ 371 Date: Apr. 28, 2021

Related U.S. Application Data
(60) Provisional application No. 62/753,379, filed on Oct. 31, 2018.

(51) Int. Cl. A61K 38/17; A61K 9/19; A61P 7/06

(52) U.S. Cl. CPC A61K 38/179; A61P 7/06; A61K 9/19

(57) ABSTRACT

Provided herein are methods for treatment of anemia due to very low, low, or intermediate risk myelodysplastic syndromes in subjects with ring sideroblasts by subcutaneous administration of an ActRIIA or ActRIIB ligand trap.

### CROSS-REFERENCE TO RELATED APPLICATIONS

This application claims the benefit of U.S. Provisional Patent Application No. 62/753,379, filed Oct. 31, 2018, which is incorporated by reference herein in its entirety.

### SEQUENCE LISTING

This application incorporates by reference in its entirety the Computer Readable Form (CRF) of a Sequence Listing in ASCII text format submitted herewith. The Sequence Listing text file submitted herewith, entitled “14247-343-228_SEQ_LISTING.txt”, was created on Oct. 29, 2019, and is 13,087 bytes in size.

### 1. FIELD

Provided herein are methods for treatment of anemia due to very low, low, or intermediate risk myelodysplastic syndromes in subjects with ring sideroblasts using, comprising administering to the subject an activin type II receptor signaling inhibitor.

### 2. BACKGROUND

Myelodysplastic syndromes (MDS) are a spectrum of hematologic malignancies characterized pathologically by the presence of morphologic dysplasia and clinically by bone marrow failure resulting in persistent and progressive cytopenias. There is considerable variation in both the clinical manifestation and severity of individual disorders within this group, ranging from the relatively mild and painless condition of refractory anemia (RA) to the much more severe refractory anemia with an excess of blasts (RAEB) that often progresses to acute leukemia (Heaney et al, N Engl J Med, 1999, 340(21):1649-60). The current treatment algorithm is based predominantly on risk stratification using the International Prognostic Scoring System (IPSS) (Greenberg et al., Blood, 1997; 89(6):2079-2088, and Erratum in Blood, 1998; 91:110). In subjects with low- or intermediate-1 (int-1) risk groups by IPSS, the goal of treatment is alleviation of cytopenias (Komrokji et al, Semin Oncol., 2011, 38(5):648-57). Subjects with MDS can be categorized into 1 of 5 risk groups (very low, low, intermediate, high, and very high) according to the International Prognostic Scoring System-Revised (IPSS-R) based on cytogenetics, hemoglobin (Hgb), platelet and absolute neutrophil count (ANC) levels, and bone marrow (BM) blast percentages obtained at diagnosis. See, e.g., Greenberg et al., Blood, 2012; 120(12):2454-2465. The 5 risk groups show significantly different risk of progression to Acute myeloid leukemia (AML) and overall survival (OS). The median survival rate is 8.8 years for subjects with low risk MDS and is as short as 0.8 years for very high-risk MDS (Greenberg et al., Blood, 2012; 120(12):2454-2465).

More than 90% of subjects diagnosed with MDS will have anemia during the course of their disease; and 30%-50% of subjects will be transfusion-dependent. Red blood cell (RBC) transfusion dependence is an independent adverse prognostic factor in MDS (Komrokji et al, Semin Oncol., 2011, 38(5):648-57).

Options for treating anemia in lower-risk MDS are limited. Erythroid-stimulating agents (ESAs) offer response rates of 20%-40%. The use of ESAs (ie, recombinant erythropoietin [EPO] or darbepoetin [DAR]) is the standard of care for low and intermediate IPSS risk patients with symptomatic anemia and an endogenous serum erythropoietin (sEPO) level<500 IU/L and is recommended by European and United States (US) treatment guidelines. The use of granulocyte-colony stimulating factor (G-CSF) may be employed as needed but is not required, although in some cases it may further improve the efficacy of the ESA (Fenaux et al., 2014, Annals of Oncology 25 (Suppl_3): iii57-iii69; Malcovati et al., 2013, Blood 122(17):2943-64) The European guidance also recommends the use of ESAs for patients who have a low RBC transfusion burden (<2 units/month) and/or an endogenous sEPO levels≤500 IU/L (Fenaux et al., 2014, Annals of Oncology 25 (Suppl_3): iii57-iii69; Malcovati et al., 2013, Blood 122(17):2943-64). However, major favorable prognostic factors for response to ESAs are a low or no RBC transfusion requirement (<2 units/month) and an endogenous sEPO level<500 IU/L (Fenaux et al., 2013, Blood; 121(21):4280-6). Responses to ESAs are best in patients with low endogenous levels (e.g., <500 IU/L) of sEPO, normal blast counts and lower IPSS/World Health Organization (WHO) Prognostic Scoring System (WPSS) scores (Hellstrom-Lindberg et al., 2003, Br J Haematol; 120(6):1037-46; Santini,V., 2011, Oncologist; 16 (supp13):35-42).

Two related type II receptors, ActRIIA and ActRIIB, have been identified as the type II receptors for activins (Mathews and Vale, 1991, Cell 65:973-982; Attisano et al., 1992, Cell 68: 97-108). Besides activins, ActRIIA and ActRIIB can biochemically interact with several other TGF-beta family proteins, including BMP7, Nodal, GDF8, and GDF11 (Yamashita et al., 1995, J. Cell Biol. 130:217-226; Lee and McPherron, 2001, Proc. Natl. Acad. Sci. 98:9306-9311; Yeo and Whitman, 2001, Mol. Cell 7: 949-957; Oh et al., 2002, Genes Dev. 16:2749-54).

Luspatercept, an ActRIIB ligand trap, has been described for treatment of various indications. See e.g. patent application publication No. 2016090077.

### 3. SUMMARY OF THE INVENTION

Provided herein is a method for treating a human subject who has been, or who is diagnosed with, anemia due to very low, low, or intermediate risk myelodysplastic syndrome (MDS), comprising administering to the subject a therapeutically effective dose of a polypeptide comprising an amino acid sequence having at least 90%, 91%, 92%, 93%, 94%, 95%, 96%, 97%, 98%, 99% or 100% sequence identity to luspatercept (a polypeptide compromising the amino acid sequence of SEQ ID NO: 1), also known as ACE-536, or sotatercept (a polypeptide compromising the amino acid sequence of SEQ ID NO: 2), also known as ACE-011, wherein (a) the subject has at least 15% of erythroblasts that are ring sideroblasts, and (b) the subject falls into one of the following groups: (i) male subjects; (ii) subjects who have received initial diagnosis of MDS between 2 to 5 years prior to said administering; (iii) subjects having a baseline platelet count higher than 100×10⁹/L, or, in a particular embodiment, 400×10⁹/L (iv) subjects having a baseline serum erythropoietin (EPO) level of between 100 to 200 IU/L; or (v) subjects who have received 4 to 6 units of RBC transfusions over the 8-week period prior to said administering.

Further provided herein is a method of treating a human subject who has been, or who is diagnosed with, very low, low, or intermediate risk MDS, or anemia due to very low, low, or intermediate risk MDS, comprising (a) determining that the subject has at least 15% of erythroblasts that are ring sideroblasts, (b) determining that the subject falls into one or more of the following groups: (i) male subjects; (ii) subjects who have received initial diagnosis of MDS between 2 to 5 years prior to said administering; (iii) subjects having a baseline platelet count higher than 100×10⁹/L, 150×10⁹/L, 200×10⁹/L, 250×10⁹/L, 300×10⁹/L, 350×10⁹/L, or, in a particular embodiment, 400×10⁹/L; (iv) subjects having a baseline serum erythropoietin (EPO) level of between 100 to 200 IU/L; or(v) subjects who have received 4 to 6 units of RBC transfusions over the 8-week period prior to said treating; and (c), on the basis of (a) and (b) combined, administering a therapeutically-effective amount of a polypeptide comprising an amino acid sequence having at least 90%, 91%, 92%, 93%, 94%, 95%, 96%, 97%, 98%, 99% or 100% sequence identity to luspatercept or sotatercept.

Provided herein is a method for treating a human subject who has been, or who is diagnosed with, anemia due to very low, low, or intermediate risk MDS, comprising administering to the subject a therapeutically effective dose of luspatercept or sotatercept, wherein (a) the subject has one or more mutations in SF3B1 gene, (b) the subject has at least 5% of erythroblasts that are ring sideroblasts, and (c) the subject falls into one of the following groups: (i) male subjects; (ii) subjects who have received initial diagnosis of MDS between 2 to 5 years prior to the administration of luspatercept or sotatercept; (iii) subjects having a baseline platelet count higher than 100×10⁹/L, 150×10⁹/L, 200×10⁹/L, 250×10⁹/L, 300×10⁹/L, 350×10⁹/L, or, in a particular embodiment, 400×10⁹/L; (iv) subjects having a baseline serum EPO level of between 100 to 200 IU/L; or (v) subjects who have received 4 to 6 units of RBC transfusions over the 8-week period prior to said administering.

Further provided herein is a method of treating a human subject who has been, or who is diagnosed with, very low, low, or intermediate risk MDS, or anemia due to very low, low, or intermediate risk MDS, comprising (a) determining that the subject has one or more mutations in SF3B1 gene (b) determining that the subject has at least 5% of erythroblasts that are ring sideroblasts, (c) determining that the subject falls into one or more of the following groups: (i) male subjects; (ii) subjects who have received initial diagnosis of MDS between 2 to 5 years prior to said administering; (iii) subjects having a baseline platelet count higher than 100×10⁹/L, 150×10⁹/L, 200×10⁹/L, 250×10⁹/L, 300×10⁹/L, 350×10⁹/L, or, in a particular embodiment, 400×10⁹/L; (iv) subjects having a baseline serum erythropoietin (EPO) level of between 100 to 200 IU/L; or (v) subjects who have received 4 to 6 units of RBC transfusions over the 8-week period prior to said treating; and (d), on the basis of (a), (b), and (c) combined, administering a therapeutically-effective amount of a polypeptide comprising an amino acid sequence having at least 90%, 91%, 92%, 93%, 94%, 95%, 96%, 97%, 98%, 99% or 100% sequence identity to luspatercept or sotatercept.

Provided herein is a method for treating a human subject who has been, or who is diagnosed with, anemia due to very low, low, or intermediate risk myelodysplastic syndrome (MDS), comprising administering to the subject a therapeutically effective dose of a polypeptide comprising an amino acid sequence having at least 90%, 91%, 92%, 93%, 94%, 95%, 96%, 97%, 98%, 99% or 100% sequence identity to SEQ ID NO: 3, SEQ ID NO: 4, or SEQ ID NO: 5, wherein (a) the subject has at least 15% of erythroblasts that are ring sideroblasts, and (b) the subject falls into one of the following groups: (i) male subjects; (ii) subjects who have received initial diagnosis of MDS between 2 to 5 years prior to said administering; (iii) subjects having a baseline platelet count higher than 100×10⁹/L, 150×10⁹/L, 200×10⁹/L, 250×10⁹/L, 300×10⁹/L, 350×10⁹/L, or, in a particular embodiment, 400×10⁹/L; (iv) subjects having a baseline serum erythropoietin (EPO) level of between 100 to 200 IU/L; or (v) subjects who have received 4 to 6 units of RBC transfusions over the 8-week period prior to said administering.

Further provided herein is a method of treating a human subject who has been, or who is diagnosed with, very low, low, or intermediate risk MDS, or anemia due to very low, low, or intermediate risk MDS, comprising (a) determining that the subject has at least 15% of erythroblasts that are ring sideroblasts, (b) determining whether the subject falls into one or more of the following groups: (i) male subjects; (ii) subjects who have received initial diagnosis of MDS between 2 to 5 years prior to said administering; (iii) subjects having a baseline platelet count higher than 100×10⁹/L, 150×10⁹/L, 200×10⁹/L, 250×10⁹/L, 300×10⁹/L, 350×10⁹/L, or, in a particular embodiment, 400×10⁹/L; (iv) subjects having a baseline serum erythropoietin (EPO) level of between 100 to 200 IU/L; or (v) subjects who have received 4 to 6 units of RBC transfusions over the 8-week period prior to said treating; and (c), on the basis of (a) and (b) combined, administering a therapeutically-effective amount of a polypeptide comprising an amino acid sequence having at least 90%, 91%, 92%, 93%, 94%, 95%, 96%, 97%, 98%, 99% or 100% sequence identity to SEQ ID NO: 3, SEQ ID NO: 4, or SEQ ID NO: 5.

Provided herein is a method for treating a human subject who has been, or who is diagnosed with, anemia due to very low, low, or intermediate risk MDS, comprising administering to the subject a therapeutically effective dose of SEQ ID NO: 3, SEQ ID NO: 4, or SEQ ID NO: 5, wherein (a) the subject has one or more mutations in SF3B1 gene, (b) the subject has at least 5% of erythroblasts that are ring sideroblasts, and (c) the subject falls into one of the following groups: (i) male subjects; (ii) subjects who have received initial diagnosis of MDS between 2 to 5 years prior to the administration of luspatercept or sotatercept; (iii) subjects having a baseline platelet count higher than 100×10⁹/L, 150×10⁹/L, 200×10⁹/L, 250×10⁹/L, 300×10⁹/L, 350×10⁹/L, or, in a particular embodiment, 400×10⁹/L; (iv) subjects having a baseline serum EPO level of between 100 to 200 IU/L; or (v) subjects who have received 4 to 6 units of RBC transfusions over the 8-week period prior to said administering.

Further provided herein is a method of treating a human subject who has been, or who is diagnosed with, very low, low, or intermediate risk MDS, or anemia due to very low, low, or intermediate risk MDS, comprising (a) determining the subject has one or more mutations in SF3B1 gene (b) determining that the subject has at least 5% of erythroblasts that are ring sideroblasts, (c) determining whether the subject falls into one or more of the following groups: (i) male subjects; (ii) subjects who have received initial diagnosis of MDS between 2 to 5 years prior to said administering; (iii) subjects having a baseline platelet count higher than 100×10⁹/L, 150×10⁹/L, 200×10⁹/L, 250×10⁹/L, 300×10⁹/L, 350×10⁹/L, or, in a particular embodiment, 400×10⁹/L; (iv) subjects having a baseline serum erythropoietin (EPO) level of between 100 to 200 IU/L; or (v) subjects who have received 4 to 6 units of RBC transfusions over the 8-week period prior to said treating; and (d), on the basis of (a), (b), and (c) combined, administering a therapeutically-effective amount of a polypeptide comprising an amino acid sequence having at least 90%, 91%, 92%, 93%, 94%, 95%, 96%, 97%, 98%, 99% or 100% sequence identity to SEQ ID NO: 3, SEQ ID NO: 4, or SEQ ID NO: 5.

Provided herein is a method for treating a human subject who has been, or who is diagnosed with, anemia due to very low, low, or intermediate risk myelodysplastic syndrome (MDS), comprising administering to the subject a therapeutically effective dose of a polypeptide comprising an amino acid sequence having at least 70%, 75%, 80%, 85%, 90%, 95%, 96%, 97%, 98%, 99% or 100% sequence identity to a fragment of SEQ ID NO: 3, SEQ ID NO: 4, or SEQ ID NO: 5, wherein (a) the subject has at least 15% of erythroblasts that are ring sideroblasts, and (b) the subject falls into one of the following groups: (i) male subjects; (ii) subjects who have received initial diagnosis of MDS between 2 to 5 years prior to said administering; (iii) subjects having a baseline platelet count higher than 100×10⁹/L, 150×10⁹/L, 200×10⁹/L, 250×10⁹/L, 300×10⁹/L, 350×10⁹/L, or, in a particular embodiment, 400×10⁹/L; (iv) subjects having a baseline serum erythropoietin (EPO) level of between 100 to 200 IU/L; or (v) subjects who have received 4 to 6 units of RBC transfusions over the 8-week period prior to said administering. In certain embodiments, the fragment of SEQ ID NO: 3 is SEQ ID NO: 5.

Further provided herein is a method of treating a human subject who has been, or who is diagnosed with, very low, low, or intermediate risk MDS, or anemia due to very low, low, or intermediate risk MDS, comprising (a) determining that the subject has at least 15% of erythroblasts that are ring sideroblasts, (b) determining whether the subject falls into one or more of the following groups: (i) male subjects; (ii) subjects who have received initial diagnosis of MDS between 2 to 5 years prior to said administering; (iii) subjects having a baseline platelet count higher than 100×10⁹/L, 150×10⁹/L, 200×10⁹/L, 250×10⁹/L, 300×10⁹/L, 350×10⁹/L, or, in a particular embodiment, 400×10⁹/L; (iv) subjects having a baseline serum erythropoietin (EPO) level of between 100 to 200 IU/L; or (v) subjects who have received 4 to 6 units of RBC transfusions over the 8-week period prior to said treating; and (c), on the basis of (a) and (b) combined, administering a therapeutically-effective amount of a polypeptide comprising an amino acid sequence having at least 70%, 75%, 80%, 85%, 90%, 95%, 96%, 97%, 98%, 99% or 100% sequence identity to a fragment of SEQ ID NO: 3, SEQ ID NO: 4, or SEQ ID NO: 5. In certain embodiments, the fragment of SEQ ID NO: 3 is SEQ ID NO: 5.

Provided herein is a method for treating a human subject who has been, or who is diagnosed with, anemia due to very low, low, or intermediate risk MDS, comprising administering to the subject a therapeutically effective dose of a fragment of SEQ ID NO: 3, SEQ ID NO: 4, or SEQ ID NO: 5, wherein (a) the subject has one or more mutations in SF3B1 gene, (b) the subject has at least 5% of erythroblasts that are ring sideroblasts, and (c) the subject falls into one of the following groups: (i) male subjects; (ii) subjects who have received initial diagnosis of MDS between 2 to 5 years prior to the administration of luspatercept or sotatercept; (iii) subjects having a baseline platelet count higher than 100×10⁹/L, 150×10⁹/L, 200×10⁹/L, 250×10⁹/L, 300×10⁹/L, 350×10⁹/L, or, in a particular embodiment, 400×10⁹/L; (iv) subjects having a baseline serum EPO level of between 100 to 200 IU/L; or (v) subjects who have received 4 to 6 units of RBC transfusions over the 8-week period prior to said administering. In certain embodiments, the fragment of SEQ ID NO: 3 is SEQ ID NO: 5.

Further provided herein is a method of treating a human subject who has been, or who is diagnosed with very low, low, or intermediate risk MDS, or anemia due to very low, low, or intermediate risk MDS, comprising (a) determining the subject has one or more mutations in SF3B1 gene (b) determining that the subject has at least 5% of erythroblasts that are ring sideroblasts, (c) determining whether the subject falls into one or more of the following groups: (i) male subjects; (ii) subjects who have received initial diagnosis of MDS between 2 to 5 years prior to said administering; (iii) subjects having a baseline platelet count higher than 100×10⁹/L, 150×10⁹/L, 200×10⁹/L, 250×10⁹/L, 300×10⁹/L, 350×10⁹/L, or, in a particular embodiment, 400×10⁹/L; (iv) subjects having a baseline serum erythropoietin (EPO) level of between 100 to 200 IU/L; or (v) subjects who have received 4 to 6 units of RBC transfusions over the 8-week period prior to said treating; and (d), on the basis of (a), (b), and (c) combined, administering a therapeutically-effective amount of a polypeptide comprising an amino acid sequence having at least 90%, 91%, 92%, 93%, 94%, 95%, 96%, 97%, 98%, 99% or 100% sequence identity to a fragment of SEQ ID NO: 3, SEQ ID NO: 4, or SEQ ID NO: 5. In certain embodiments, the fragment of SEQ ID NO: 3 is SEQ ID NO: 5.

In certain embodiments of any of the foregoing methods, the very low, low, or intermediate risk MDS is categorized using International Prognostic Scoring System-Revised (IPS S-R).

In certain embodiments of any of the foregoing methods, the subject has less than 5 percent of blasts in bone marrow.

In certain embodiments of any of the foregoing methods, the subject is a subject requiring red blood cell (RBC) transfusion.

In certain embodiments of any of the foregoing methods, the method is a method to achieve (i) a long-term, or more specifically, 8-weeks or longer, reduction in a percentage of erythroblasts in the subject that are ring sideroblasts as compared to an initial percentage of erythroblasts in the subject that are ring sideroblasts; and/or (ii) a long-term, or more specifically, 8-weeks or longer increase in hemoglobin level in the subject as compared to the hemoglobin level in the subject a period of time prior to administering to the subject an initial dose of said administering.

In certain embodiments of any of the foregoing methods, the percentage of erythroblasts in the subject are ring sideroblasts prior to said administering, e.g., the administering of luspatercept or sotatercept, is at least 15%, 16%, 17%, 18%, 19%, or at least 20%. In certain embodiments, the subject treated prior to said administering has at least 15% of erythroblasts in the subject are ring sideroblasts. In certain embodiments, the subject treated prior to said administering (i) has one or more mutations in SF3B1 gene, and (ii) has at least 5%, 6%, 7%, 8%, 9%, 10%, 11%, 12%, 13%, 14%, 15%, 16%, 17%, 18%, 19%, or at least 20% of erythroblasts that are ring sideroblasts.

In certain embodiments of any of the foregoing methods, a pharmaceutically effective dose of luspatercept or sotatercept is between 1.0 mg/kg and 1.75 mg/kg. In certain embodiments of any of the foregoing methods, the pharmaceutically effective dose of luspatercept or sotatercept is 0.45 mg/kg, 0.50 mg/kg, 0.60 mg/kg, 0.70 mg/kg, 0.80 mg/kg, 0.90 mg/kg, 1.00 mg/kg, 1.05 mg/kg, 1.10 mg/kg, 1.15 mg/kg, 1.20 mg/kg, 1.25 mg/kg, 1.30 mg/kg, 1.33 mg/kg, 1.35 mg/kg, 1.40 mg/kg, 1.45 mg/kg, 1.50 mg/kg, 1.55 mg/kg, 1.60 mg/kg, 1.65 mg/kg, 1.70 mg/kg, or 1.75 mg/kg.

In certain embodiments of any of the foregoing methods, luspatercept or sotatercept is administered subcutaneously.

In certain embodiments of any of the foregoing methods, a subject can be refractory to prior erythropoiesis-stimulating agents (ESA) treatment. In certain embodiments of any of the foregoing methods, a subject can be intolerant to prior ESA treatment. In certain embodiments of any of the foregoing methods, a subject can be ineligible to ESA treatment.

In certain embodiments of any of the foregoing methods, a subject who is refractory to prior ESA treatment can be a subject who has a non-response or is no longer responsive to prior ESA-containing regimen, either as single agent or combination with other agent, at any time after introduction due to intolerance or an adverse event.

In certain embodiments of any of the foregoing methods, the subject is intolerant to prior ESA treatment. In certain embodiments, the prior ESA-containing regimen, either as single agent or combination with other agent, at any time after introduction has been discontinued in the subject due to intolerance or an adverse event.

In certain embodiments of any of the foregoing methods, the subject has a low chance to respond to ESA treatments due to a high endogenous serum erythropoietin (EPO) level. In certain embodiments of any of the foregoing methods, the subject has not been previously treated with ESAs and has a serum EPO level >200 IU/L.

In certain embodiments of any of the foregoing methods, the ESA-containing regimen also contains granulocyte-colony stimulating factor (G-CSF).

In certain embodiments, the outcome of any of the above methods is: (a) the subject treated has a duration for red blood cell transfusion independence (RBC-TI) greater than or equal to 8 weeks after said administering; (b) the subject treated has RBC-TI greater than or equal to 12 weeks; or (c) the subject treated has a modified erythroid response (mHI-E). In certain embodiments, the mHI-E is a mean hemoglobin increase of greater than or equal to 1.5 g/dL over 8 weeks, or reduction of 4 or more units of red blood cells transfused over 8 weeks, after said administering.

### 4. BRIEF DESCRIPTION OF THE DRAWINGS

FIG. 1 depicts demographic and baseline characteristics of the intent-to-treat (ITT) population.

FIG. 2 depicts prior transfusion history of the ITT population.

FIG. 3 summarizes the medical history data using frequency tabulations by the Medical Dictionary for Regulatory Activities (MedDRA) system organ class and preferred term for the ITT population.

FIG. 4 depicts the prior history of MDS diagnoses using frequency tabulations for the ITT population.

FIG. 5 depicts RBC transfusion dependences using frequency tabulations.

FIG. 6 depicts the prior medical history of ESA treatments, GCSF/GMCSF usages, and iron chelation therapy treatments of the ITT population.

FIG. 7A depicts part A of the forest plot of a subgroup analysis for RBC-TI equal to or more than 8 weeks during weeks 1-24 for the ITT population.

FIG. 7B depicts part B of the forest plot of a subgroup analysis for RBC-TI equal to or more than 8 weeks during weeks 1-24 for the ITT population.

FIG. 8A depicts part A of the forest plot of a subgroup analysis for RBC-TI equal to or more than 12 weeks during weeks 1-24 for the ITT population.

FIG. 8B depicts part B of the forest plot of a subgroup analysis for RBC-TI equal to or more than 12 weeks during weeks 1-24 for the ITT population.

FIG. 9A depicts part A of the forest plot of a subgroup analysis for RBC-TI equal to or more than 12 weeks during weeks 1-48 for the ITT population.

FIG. 9B depicts part B of the forest plot of a subgroup analysis for RBC-TI equal to or more than 12 weeks during weeks 1-48 for the ITT population.

FIG. 10A depicts part A of the forest plot of a subgroup analysis for HI-E during weeks 1-24 for the ITT population.

FIG. 10B depicts part B of the forest plot of a subgroup analysis for HI-E during weeks 1-24 for the ITT population.

FIG. 11A depicts part A of the forest plot of a subgroup analysis for HI-E during weeks 1-48 for the ITT population.

FIG. 11B depicts part A of the forest plot of a subgroup analysis for HI-E during weeks 1-48 for the ITT population.

### 5. DETAILED DESCRIPTION OF THE INVENTION

### 5.1 Overview

Provided herein are methods of treating anemia due to very low, low, or intermediate risk Myelodysplastic syndromes (MDS) in subjects with ring sideroblasts using activin-ActRIIA or activin-ActRIIB ligand traps. Statistical analysis was performed on the intent-to-treat (ITT) population to demonstrate efficacy, including subgroups analysis by age, gender, race, baseline characteristics (e.g. baseline serum EPO level), medical history (e.g. time since initial diagnosis), and baseline RBC transfusion dependency. See section 6.1.2.

### 5.2 Terms and Abbreviations

As used herein, “ActRII” refers to activin receptor type II. As used herein, “ActRIIA” refers to activin receptor type IIA. See, for example, Mathews and Vale, 1991, Cell 65:973-982. GenBank™ accession number NM_001278579.1 provides an exemplary human ActRIIA nucleic acid sequence. GenBank™ accession number NP_001265508.1 provides an exemplary human ActRIIA amino acid sequence.

As used herein, “ActRIIB” refers to activin receptor type IIB. See, for example, Attisano et al., 1992, Cell 68: 97-108. GenBank™ accession number NM_001106.3 provides an exemplary human ActRIIB nucleic acid sequence. GenBank™ accession number NP_001097.2 provides an exemplary human ActRIIB amino acid sequence.

As used herein, “BL” refers to baseline.

As used herein, “DAR” refers to darbepoetin.

As used herein, “ECD” refers to extracellular domain.

As used herein, “EPO” refers to erythropoietin.

As used herein, “sEPO” refers to serum erythropoietin.

As used herein, “ESA” refers to erythropoiesis-stimulating agent.

As used herein, “G-CSF” refers to granulocyte colony-stimulating factor.

As used herein, “GM-CSG” refers to granulocyte macrophage colony-stimulating factor.

As used herein, “Hb” refers to hemoglobin.

As used herein, “HI-E” refers to erythroid hematological improvement. In certain embodiments, the HI-E is as defined by IWG. In certain embodiments, the HI-E is as defined by the modified 2006 IWG. In certain embodiments, the HI-E for a low transfusion burden patient is an increase in hemoglobin concentration in the patient of at least 1.5 g/dL for at least 8 weeks. In certain embodiments, the HI-E for a high transfusion burden patient is an at least 4 unit reduction in RBC transfusion over 8 weeks.

As used herein, “HTB” refers to high transfusion burden. In certain embodiments, a HTB subject receives greater than or equal to 4 RBC units over the course of 8 weeks.

As used herein, “IgG” refers to immunoglobulin G.

As used herein, “IPSS-R” refers to International Prognostic Scoring System-Revised. See section 5.4.

As used herein, “IWG” refers to International Working Group. See, e.g., Cheson et al. Blood. 2000 96:3671-3674. In certain embodiments, IWG refers to the modified 2006 criteria. See, e.g., Cheson et al., 2006, Blood, 108(2).

As used herein, “LTB” refers to low transfusion burden. In certain embodiments, a LTB subject receives less than 4 RBC units over the course of 8 weeks.

As used herein, “ITT” refers to intent-to-treat.

As used herein, “MedDRA” refers to Medical Dictionary for Regulatory Activities.

As used herein, “MDS” refers to myelodysplastic syndromes.

As used herein, “PD” refers to pharmacodynamic.

As used herein, “PK” refers to pharmacokinetic.

As used herein, “RA” refers to refractory anemia.

As used herein, “RAEB” refers to refractory anemia with an excess of blasts.

As used herein, “RBC” refers to red blood cells.

As used herein, “RBC-TI” refers to red blood cell transfusion independent.

As used herein, “RCMD-RS” refers to refractory cytopenia with multilineage dysplasia with ring sideroblasts.

As used herein, “RS” refers to ring sideroblast.

As used herein, “SC” refers to subcutaneous.

As used herein, “SF3B1” refers to splicing factor 3B1. GenBank™ accession numbers NM_012433.3, NM_001005523.2, and NM_001308824.1 provide exemplary nucleic acid sequences for human SF3B1. GenBank™ accession numbers NP_001295753.1, NP_001005526.1, and NP_036565.2 provide exemplary amino acid sequences for human SF3B1.

As used herein, “WPSS” refers to World Health Organization (WHO) Prognostic Scoring System.

As used herein, “luspatercept” refers to a polypeptide comprising the amino acid sequence of SEQ ID: NO 1.

As used herein, “sotatercept” refers to a polypeptide comprising the amino acid sequence of SEQ ID: NO 2.

### 5.3 Methods of Treatment

In one aspect, provided herein is a method of treating, very low, low, or intermediate risk MDS, or anemia due to very low, low, or intermediate risk MDS, in a subject, comprising administering a pharmaceutically effective dose of an ActRII signaling inhibitor (between 0.1 mg/kg and 2.0 mg/kg) to the subject if at least 5%, 6%, 7%, 8%, 9%, 10%, 11%, 12%, 13%, 14%, 15%, 16%, 17%, 18%, 19%, or 20% of erythroblasts in the subject are ring sideroblasts. See section 5.4 for patient population that can be treated with the method provided herein and subpopulations. In certain embodiments, the subject has one or more mutations in SF3B1 gene. In certain embodiments, the percentage of erythroblasts in the subject that are ring sideroblasts is determined at a first time. In certain embodiments, the first time is a within 1 day, 2 days, 3, days, 4 days, 5 days, 6 days, 1 week, 2 weeks, 3 weeks, 4 weeks, 5 weeks, 6 weeks, 7 weeks, 8 weeks, 3 months, 4 months, 5 months, 6 months, 1 year, 2 years, 3 years, 4 years, and 5 years after administering the pharmaceutically effective dose of the ActRII signaling inhibitor to the subject.

Provided herein is a method of treating, very low, low, or intermediate risk MDS, or anemia due to very low, low, or intermediate risk MDS, in a subject, comprising administering to the subject an activin receptor type II (ActRII) signaling inhibitor at a pharmaceutically effective dose and for a period of time to achieve (i) a long-term reduction in a percentage of erythroblasts in the subject that are ring sideroblasts as compared to an initial percentage of erythroblasts in the subject that are ring sideroblasts; and/or (ii) a long-term increase in hemoglobin level in the subject as compared to the hemoglobin level in the subject a period of time prior to administering to the subject an initial dose of the ActRII signaling inhibitor; wherein the pharmaceutically effective dose is between 0.1 mg/kg and 2.0 mg/kg, and wherein the initial percentage of erythroblasts in the subject that are ring sideroblasts is at least 5%, 6%, 7%, 8%, 9%, 10%, 11%, 12%, 13%, 14%, 15%, 16%, 17%, 18%, 19%, or at least 20%.

In certain embodiments, the ActRII signaling inhibitor is administered once every 1, 2, 3, 4, 5, or 6 weeks. In certain embodiments, the ActRII signaling inhibitor is administered once every 21 days. In certain embodiments, the ActRII signaling inhibitor is administered subcutaneously.

In certain embodiments, the ActRII signaling inhibitor is luspatercept (a polypeptide comprising the amino acid sequence of SEQ ID NO: 1). In certain embodiments, the ActRII signaling inhibitor is sotatercept (a polypeptide comprising the amino acid sequence of SEQ ID NO: 2). In certain embodiments, the ActRII signaling inhibitor is a polypeptide comprising the amino acid sequence of SEQ ID NO: 3. In certain embodiments, the ActRII signaling inhibitor is a polypeptide comprising a fragment of the amino acid sequence of SEQ ID NO: 3. In certain embodiments, the ActRII signaling inhibitor is a polypeptide comprising the amino acid sequence of SEQ ID NO: 4. In certain embodiments, the ActRII signaling inhibitor is a polypeptide comprising a fragment of the amino acid sequence of SEQ ID NO: 4. In certain embodiments, the ActRII signaling inhibitor is a polypeptide comprising the amino acid sequence of SEQ ID NO: 5. In certain embodiments, the ActRII signaling inhibitor is a humanized fusion-protein consisting of the extracellular domain of ActRIIA and the human IgG1 Fc domain. In certain embodiments, the ActRII signaling inhibitor is a humanized fusion-protein consisting of the extracellular domain of ActRIIB and the human IgG1 Fc domain.

In certain embodiments, the ActRII signaling inhibitor is a signaling inhibitor of ActRIIB In certain embodiments, the ActRIIB signaling inhibitor is a polypeptide comprising an amino acid sequence selected from the group consisting of: (a) 90% identical to SEQ ID NO: 1; (b) 91% identical to SEQ ID NO: 1; (c) 92% identical to SEQ ID NO: 1; (d) 93% identical to SEQ ID NO: 1; (e) 94% identical to SEQ ID NO: 1; (f) 95% identical to SEQ ID NO: 1; (g) 96% identical to SEQ ID NO: 1; (h) 97% identical to SEQ ID NO: 1; (i) 98% identical to SEQ ID NO: 1; (j) 99% identical to SEQ ID NO: 1; and (k) 100% identical to SEQ ID NO: 1.

In certain embodiments, the ActRII signaling inhibitor is a signaling inhibitor of ActRIIA. In certain embodiments, the ActRIIA signaling inhibitor is a polypeptide comprising an amino acid sequence selected from the group consisting of: (a) 90% identical to SEQ ID NO: 2; (b) 91% identical to SEQ ID NO: 2; (c) 92% identical to SEQ ID NO: 2; (d) 93% identical to SEQ ID NO: 2; (e) 94% identical to SEQ ID NO: 2; (f) 95% identical to SEQ ID NO: 2; (g) 96% identical to SEQ ID NO: 2; (h) 97% identical to SEQ ID NO: 2; (i) 98% identical to SEQ ID NO: 2; (j) 99% identical to SEQ ID NO: 2; and (k) 100% identical to SEQ ID NO: 2.

In certain embodiments, the ActRII signaling inhibitor is a signaling inhibitor of ActRIIB In certain embodiments, the ActRIIB signaling inhibitor is a polypeptide comprising an amino acid sequence selected from the group consisting of: (a) 90% identical to SEQ ID NO: 3; (b) 91% identical to SEQ ID NO: 3; (c) 92% identical to SEQ ID NO: 3; (d) 93% identical to SEQ ID NO: 3; (e) 94% identical to SEQ ID NO: 3; (f) 95% identical to SEQ ID NO: 3; (g) 96% identical to SEQ ID NO: 3; (h) 97% identical to SEQ ID NO: 3; (i) 98% identical to SEQ ID NO: 3; (j) 99% identical to SEQ ID NO: 3; and (k) 100% identical to SEQ ID NO: 3.

In certain embodiments, the ActRII signaling inhibitor is a signaling inhibitor of ActRIIA. In certain embodiments, the ActRIIA signaling inhibitor is a polypeptide comprising an amino acid sequence selected from the group consisting of: (a) 90% identical to SEQ ID NO: 4; (b) 91% identical to SEQ ID NO: 4; (c) 92% identical to SEQ ID NO: 4; (d) 93% identical to SEQ ID NO: 4; (e) 94% identical to SEQ ID NO: 4; (f) 95% identical to SEQ ID NO: 4; (g) 96% identical to SEQ ID NO: 4; (h) 97% identical to SEQ ID NO: 4; (i) 98% identical to SEQ ID NO: 4; (j) 99% identical to SEQ ID NO: 4; and (k) 100% identical to SEQ ID NO: 4.

In certain embodiments, the ActRII signaling inhibitor is a signaling inhibitor of ActRIIB In certain embodiments, the ActRIIB signaling inhibitor is a polypeptide comprising an amino acid sequence selected from the group consisting of: (a) 90% identical to SEQ ID NO: 5; (b) 91% identical to SEQ ID NO: 5; (c) 92% identical to SEQ ID NO: 5; (d) 93% identical to SEQ ID NO: 5; (e) 94% identical to SEQ ID NO: 5; (f) 95% identical to SEQ ID NO: 5; (g) 96% identical to SEQ ID NO: 5; (h) 97% identical to SEQ ID NO: 5; (i) 98% identical to SEQ ID NO: 5; (j) 99% identical to SEQ ID NO: 5; and (k) 100% identical to SEQ ID NO: 5.

In certain embodiments, the ActRII signaling inhibitor is a signaling inhibitor of ActRIIB In certain embodiments, the ActRIIB signaling inhibitor is a polypeptide comprising an amino acid sequence selected from the group consisting of: (a) 70% identical to a fragment of SEQ ID NO: 3; (b) 71% identical to a fragment of SEQ ID NO: 3; (c) 72% identical to a fragment of SEQ ID NO: 3; (d) 73% identical to a fragment of SEQ ID NO: 3; (e) 74% identical to a fragment of SEQ ID NO: 3; (f) 75% identical to a fragment of SEQ ID NO: 3; (g) 76% identical to a fragment of SEQ ID NO: 3; (h) 77% identical to a fragment of SEQ ID NO: 3; (i) 78% identical to a fragment of SEQ ID NO: 3; (j) 79% identical to a fragment of SEQ ID NO: 3; (k) 80% identical to a fragment of SEQ ID NO: 3; (1) 81% identical to a fragment of SEQ ID NO: 3; (m) 82% identical to a fragment of SEQ ID NO: 3; (n) 83% identical to a fragment of SEQ ID NO: 3; (o) 84% identical to a fragment of SEQ ID NO: 3; (p) 85% identical to a fragment of SEQ ID NO: 3; (q) 86% identical to a fragment of SEQ ID NO: 3; (r) 87% identical to a fragment of SEQ ID NO: 3; (s) 88% identical to a fragment of SEQ ID NO: 3; (t) 89% identical to a fragment of SEQ ID NO: 3; (u) 90% identical to a fragment of SEQ ID NO: 3; (v) 91% identical to a fragment of SEQ ID NO: 3; (w) 92% identical to a fragment of SEQ ID NO: 3; (x) 93% identical to a fragment of SEQ ID NO: 3; (y) 94% identical to a fragment of SEQ ID NO: 3; (z) 95% identical to a fragment of SEQ ID NO: 3; (aa) 96% identical to a fragment of SEQ ID NO: 3; (ab) 97% identical to a fragment of SEQ ID NO: 3; (ac) 98% identical to a fragment of SEQ ID NO: 3; (ad) 99% identical to a fragment of SEQ ID NO: 3; and (ae) 100% identical to a fragment of SEQ ID NO: 3.

In certain embodiments, the ActRII signaling inhibitor is a signaling inhibitor of ActRIIA. In certain embodiments, the ActRIIA signaling inhibitor is a polypeptide comprising an amino acid sequence selected from the group consisting of: (a) 70% identical to a fragment of SEQ ID NO: 4; (b) 71% identical to a fragment of SEQ ID NO: 4; (c) 72% identical to a fragment of SEQ ID NO: 4; (d) 73% identical to a fragment of SEQ ID NO: 4; (e) 74% identical to a fragment of SEQ ID NO: 4; (f) 75% identical to a fragment of SEQ ID NO: 4; (g) 76% identical to a fragment of SEQ ID NO: 4; (h) 77% identical to a fragment of SEQ ID NO: 4; (i) 78% identical to a fragment of SEQ ID NO: 4; (j) 79% identical to a fragment of SEQ ID NO: 4; (k) 80% identical to a fragment of SEQ ID NO: 4; (l) 81% identical to a fragment of SEQ ID NO: 4; (m) 82% identical to a fragment of SEQ ID NO: 4; (n) 83% identical to a fragment of SEQ ID NO: 4; (o) 84% identical to a fragment of SEQ ID NO: 4; (p) 85% identical to a fragment of SEQ ID NO: 4; (q) 86% identical to a fragment of SEQ ID NO: 4; (r) 87% identical to a fragment of SEQ ID NO: 4; (s) 88% identical to a fragment of SEQ ID NO: 4; (t) 89% identical to a fragment of SEQ ID NO: 4; (u) 90% identical to a fragment of SEQ ID NO: 4; (v) 91% identical to a fragment of SEQ ID NO: 4; (w) 92% identical to a fragment of SEQ ID NO: 4; (x) 93% identical to a fragment of SEQ ID NO: 4; (y) 94% identical to a fragment of SEQ ID NO: 4; (z) 95% identical to a fragment of SEQ ID NO: 4; (aa) 96% identical to a fragment of SEQ ID NO: 4; (ab) 97% identical to a fragment of SEQ ID NO: 4; (ac) 98% identical to a fragment of SEQ ID NO: 4; (ad) 99% identical to a fragment of SEQ ID NO: 4; and (ae) 100% identical to a fragment of SEQ ID NO: 4.

In certain embodiments, the ActRII signaling inhibitor is a signaling inhibitor of ActRIIB In certain embodiments, the ActRIIB signaling inhibitor is a polypeptide comprising an amino acid sequence selected from the group consisting of: (a) 70% identical to a fragment of SEQ ID NO: 5; (b) 71% identical to a fragment of SEQ ID NO: 5; (c) 72% identical to a fragment of SEQ ID NO: 5; (d) 73% identical to a fragment of SEQ ID NO: 5; (e) 74% identical to a fragment of SEQ ID NO: 5; (f) 75% identical to a fragment of SEQ ID NO: 5; (g) 76% identical to a fragment of SEQ ID NO: 5; (h) 77% identical to a fragment of SEQ ID NO: 5; (i) 78% identical to a fragment of SEQ ID NO: 5; (j) 79% identical to a fragment of SEQ ID NO: 5; (k) 80% identical to a fragment of SEQ ID NO: 5; (l) 81% identical to a fragment of SEQ ID NO: 5; (m) 82% identical to a fragment of SEQ ID NO: 5; (n) 83% identical to a fragment of SEQ ID NO: 5; (o) 84% identical to a fragment of SEQ ID NO: 5; (p) 85% identical to a fragment of SEQ ID NO: 5; (q) 86% identical to a fragment of SEQ ID NO: 5; (r) 87% identical to a fragment of SEQ ID NO: 5; (s) 88% identical to a fragment of SEQ ID NO: 5; (t) 89% identical to a fragment of SEQ ID NO: 5; (u) 90% identical to a fragment of SEQ ID NO: 5; (v) 91% identical to a fragment of SEQ ID NO: 5; (w) 92% identical to a fragment of SEQ ID NO: 5; (x) 93% identical to a fragment of SEQ ID NO: 5; (y) 94% identical to a fragment of SEQ ID NO: 5; (z) 95% identical to a fragment of SEQ ID NO: 5; (aa) 96% identical to a fragment of SEQ ID NO: 5; (ab) 97% identical to a fragment of SEQ ID NO: 5; (ac) 98% identical to a fragment of SEQ ID NO: 5; (ad) 99% identical to a fragment of SEQ ID NO: 5; and (ae) 100% identical to a fragment of SEQ ID NO: 5.

In certain embodiments, the dose of the ActRII signaling inhibitor is between 0.1 and 2.25 mg/kg. In certain embodiments, the dose of the ActRII signaling inhibitor is between 0.1 and 2.0 mg/kg. In certain embodiments, the dose of the ActRII signaling inhibitor is between 0.7 and 2.0 mg/kg. In certain embodiments, the dose of the ActRII signaling inhibitor is about 0.1 mg/kg, 0.125 mg/kg, 0.3 mg/kg, 0.5 mg/kg, 0.7 mg/kg, 1.0 mg/kg, 1.25 mg/kg, 1.33 mg/kg, 1.5 mg/kg, 1.75 mg/kg, 2.0 mg/kg, or 2.25 mg/kg. In certain embodiments, the dose of the ActRII signaling inhibitor is between 0.1 mg/kg and 0.5 mg/kg, between 0.3 mg/kg and 0.7 mg/kg, between 0.5 mg/kg and 1.0 mg/kg, between 0.7 mg/kg and 1.25 mg/kg, between 1.0 mg/kg and 2.0 mg/kg, or between 1.5 and 2.25 mg/kg.

In certain embodiments, the ActRIl signaling inhibitor is administered once every 21 days. In certain embodiments, the ActRIl signaling inhibitor is administered subcutaneously. In certain embodiments, the treatment duration is a maximum of 24 months. In certain embodiments, the maximum total dose per administration is below 168 mg.

In certain embodiments, the outcome of any of the above methods is: (a) the subject treated has a duration for red blood cell transfusion independence (RBC-TI) greater than or equal to 8 weeks after said administering; (b) the subject treated has RBC-TI greater than or equal to 12 weeks; or (c) the subject treated has a modified erythroid response (mHI-E). In certain embodiments, the mHI-E is a mean hemoglobin increase of greater than or equal to 1.5 g/dL over 8 weeks, or reduction of 4 or more units of red blood cells transfused over 8 weeks, after said administering.

### 5.4 Patient Populations

The subjects treated in accordance with the methods described herein can be any mammals such as rodents, domestic animals such as dogs or cats, or primates, e.g. non-human primates. In a preferred embodiment, the subject is a human. In certain embodiments, the methods described herein can be used to treat anemia due to very low, low, or intermediate risk myelodysplastic syndromes (MDS) in a subject; to reduce transfusion burden in a subject with anemia, or to monitor said treatment; and/or to select subjects to be treated in accordance with the methods provided herein, in any mammal such as a rodent or primate, and in a preferred embodiment, in a human subject.

In certain embodiments, the subject treated in accordance with the methods described herein is female. In certain embodiments, the subject treated in accordance with the methods described herein is male. In certain embodiments, the subject treated in accordance with the methods described herein can be of any age. In certain embodiments, the subject treated in accordance with the methods described herein is less than 18 years old. In a specific embodiment, the subject treated in accordance with the methods described herein is less than 13 years old. In another specific embodiment, the subject treated in accordance with the methods described herein is less than 12, less than 11, less than 10, less than 9, less than 8, less than 7, less than 6, or less than 5 years old. In another specific embodiment, the subject treated in accordance with the methods described herein is 1-3 years old, 3-5 years old, 5-7 years old, 7-9 years old, 9-11 years old, 11-13 years old, 13-15 years old, 15-20 years old, 20-25 years old, 25-30 years old, or greater than 30 years old. In another specific embodiment, the subject treated in accordance with the methods described herein is 30-35 years old, 35-40 years old, 40-45 years old, 45-50 years old, 50-55 years old, 55-60 years old, or greater than 60 years old. In another specific embodiment, the subject treated in accordance with the methods described herein is 18-64 years old, 65-74 years old, or greater than 75 years old.

In certain embodiments, a subject treated in accordance with the methods provided herein has been diagnosed with IPSS-R defined MDS. IPSS-R refers to the International Prognostic Scoring System-Revised, which is utilized in the evaluation of prognosis in myelodysplastic syndromes. See, e.g., Greenberg et al., Blood, 2012; 120(12):2454-2465. The IPSS-R utilizes a criteria point system to characterize myelodysplastic syndrome patient outcomes as very low risk (0-1.5 risk score, median survival 8.8 years), low risk (1.5-3.0 risk score; median survival of 5.3 years), intermediate (3.0-4.5 point; median survival of 3.0 years); high risk (4.5-6.0 points; median survival of 1.6 years); or very high risk (risk score higher than 6; median survival of 0.8 years). The point system evaluates (i) the percentage of bone marrow blasts in the subject; and (ii) cytogenetics in the subject which defined as hemoglobin concentration (g/dL), absolute neutrophil count (×10⁹/L), and platelet count (×10⁹/L).

In certain embodiments, a subject treated in accordance with the methods provided herein has MDS. In certain embodiments, the MDS is IPSS-defined very low risk MDS. In certain embodiments, the MDS is IPSS-R defined low risk MDS. In certain embodiments, the MDS is IPSS-R defined intermediate risk MDS. In certain embodiments, a subject treated in accordance with the methods provided herein has MDS-refractory cytopenia with multilineage dysplasia (MDS-RCMD).

In certain embodiments, the subject treated in accordance with the methods described herein has an Eastern Cooperative Oncology Group (ECOG) score of 0. In certain embodiments, the subject treated in accordance with the methods described herein has an ECOG score of 1. In certain embodiments, the subject treated in accordance with the methods described herein has an ECOG score of 2.

In certain embodiments, the percentage of erythroblasts in a subject treated in accordance with the methods provided herein that are ring sideroblasts is at least 5%, 6%, 7%, 8%, 9%, 10%, 11%, 12%, 13%, 14%, 15%, 16%, 17%, 18%, 19%, or at least 20%. In certain embodiments, the percentage of erythroblasts in a subject treated in accordance with the methods provided herein that are ring sideroblasts is at least 15%. In certain embodiments, the percentage of erythroblasts in a subject treated in accordance with the methods provided herein that are ring sideroblasts is about 15%. In certain embodiments, the percentage of erythroblasts in a subject treated in accordance with the methods provided herein that are ring sideroblasts is between about 15% and about 20%. In certain embodiments, the percentage of erythroblasts in a subject treated in accordance with the methods provided herein that are ring sideroblasts is between about 5% and 20%. In certain embodiments, a subject treated in accordance with the methods provided herein has a ringed sideroblast to normal erythroblast ratio of at least 1:20, at least 1:7, or at least 1:5.

In certain embodiments, a subject having anemia due to very low, low, or intermediate risk MDS treated requires regular, lifelong red blood cell transfusions. In certain embodiments, a subject having anemia due to very low, low, or intermediate risk MDS requires transfusion of 0 to 4 red blood cell units over a 8-weeks period. In certain embodiments, a subject having anemia due to very low, low, or intermediate risk MDS requires transfusion of 4 to 6 red blood cell units over a 8-weeks period. In certain embodiments, a subject having anemia due to very low, low, or intermediate risk MDS requires transfusion of less than 6 red blood cell units over a 8-weeks period. In certain embodiments, a subject having anemia due to very low, low, or intermediate risk MDS requires transfusion of more than 6 red blood cell units over a 8-weeks period. In certain embodiments, a subject having anemia due to very low, low, or intermediate risk MDS has a high transfusion burden. In certain embodiments, high transfusion burden is 12 or more red blood cell units over 24 weeks prior to treatment according to the methods provided herein. In certain embodiments, a subject treated in accordance with the methods provided herein has a low transfusion burden. In certain embodiments, the subject with a low transfusion burden treated in accordance with the methods provided herein requires at most 0, 1, 2, or 3 units of red blood cells per 8 weeks. In certain embodiments, a subject treated in accordance with the methods provided herein has a high transfusion burden. In certain embodiments, the subject with a high transfusion burden treated in accordance with the methods provided herein requires at least 4, 5, 6, 7, 8, 9, 10, 11, 12, or 13 units of red blood cells per 8 weeks.

In certain embodiments, a subject treated has one or more mutations in the SF3B1 gene. In certain embodiments, the one or more mutations in SF3B1 gene has been confirmed by genetic analysis. In certain embodiments, the one or more mutations is in a non-coding region. In certain embodiments, SF3B1 is the gene encoding SB3B1. In certain embodiments, the one or more mutations is in a coding region. In certain embodiments, SF3B1 is SF3B1 protein. In certain embodiments, the one or more mutations in SF3B1 protein is selected from the group consisting of E622D, R625C, H662Q, H662D, K66N, K666T, K666Q, K666E, A672D, K700E, I704N. In certain embodiments, a subject treated in accordance with the methods provided herein expresses SF3B1 protein with the mutation E622D. In certain embodiments, a subject treated in accordance with the methods provided herein expresses SF3B1 protein with the mutation R625C. In certain embodiments, a subject treated in accordance with the methods provided herein expresses SF3B1 protein with the mutation H662Q. In certain embodiments, a subject treated in accordance with the methods provided herein expresses SF3B1 protein with the mutation H662D. In certain embodiments, a subject treated in accordance with the methods provided herein expresses SF3B1 protein with the mutation K66N. In certain embodiments, a subject treated in accordance with the methods provided herein expresses SF3B1 protein with the mutation K666T. In certain embodiments, a subject treated in accordance with the methods provided herein expresses SF3B1 protein with the mutation K666Q. In certain embodiments, a subject treated in accordance with the methods provided herein expresses SF3B1 protein with the mutation K666E. In certain embodiments, a subject treated in accordance with the methods provided herein expresses SF3B1 protein with the mutation A672D. In certain embodiments, a subject treated in accordance with the methods provided herein expresses SF3B1 with the mutation K700E. In certain embodiments, a subject treated in accordance with the methods provided herein expresses SF3B1 protein with the mutation I704N. In a specific embodiment, a subject treated in accordance with the methods provided herein expresses SRSF2 with one or more mutations. In a specific embodiment, a subject treated in accordance with the methods provided herein expresses DNMT3A with one or more mutations. In a specific embodiment, a subject treated in accordance with the methods provided herein expresses TET2 with one or more mutations. In a specific embodiment, a subject treated in accordance with the methods provided herein expresses SETBP1 with one or more mutations.

In certain embodiments, a subject treated in accordance with the methods provided herein (i) has anemia due to very low, low or intermediate risk MDS, (ii) at least 15% of erythroblasts in the subject are ring sideroblasts. In certain embodiments, a subject treated in accordance with the methods provided herein (i) has anemia due to very low, low or intermediate risk MDS, (ii) at least 5% of erythroblasts in the subject are ring sideroblasts, and (iii) expresses SF3B1 with one or more mutations.

In certain embodiments, a subject treated in accordance with the methods provided herein has thrombocytopenia. In certain embodiments, a subject treated in accordance with the methods provided herein has less than 100×10⁹ platelets per liter. In certain embodiments, a subject treated in accordance with the methods provided herein has 100 to 400×10⁹ platelets per liter. In certain embodiments, a subject treated in accordance with the methods provided herein has more than 400×10⁹ platelets per liter. In certain embodiments, a subject treated in accordance with the methods provided herein has neutropenia. In certain embodiments, a subject treated in accordance with the methods provided herein has an absolute neutrophil count of less than 1×10⁹ per liter.

In certain embodiments, a subject treated in accordance with the methods provided herein has less than 13,000 white blood cells per μL, less than 12,000 white blood cells per μL, less than 11,000 white blood cells per μL, less than 10,000 white blood cells per μL, less than 7,500 white blood cells per μL, or less than 500 white blood cells per μL.

In certain embodiments, hemoglobin levels in a subject treated in accordance with the methods provided herein are less than 10 g/dL, 9 g/dL, 8 g/dL, or 7 g/dL. In certain embodiments, hemoglobin levels in a subject treated in accordance with the methods provided herein are between 7 g/dL and 7.5 g/dL, between 7.5 g/dL and 8 g/dL, between 8 g/dL and 8.5 g/dL, between 8.5 g/dL and 9.0 g/dL, between 9.0 g/dL and 9.5 g/dL, or between 9.5 g/dL and 10.0 g/dL.

In certain embodiments of any of the foregoing methods, a subject can be refractory to prior Erythropoiesis-stimulating agents (ESA) treatment. In certain embodiments of any of the foregoing methods, a subject can be intolerant to prior ESA treatment. In certain embodiments of any of the foregoing methods, a subject can be ineligible to ESA treatment.

In certain embodiments of any of the foregoing methods, a subject who is refractory to prior ESA treatment can be a subject who has a non-response or is no longer responsive to prior ESA-containing regimen, either as single agent or combination with other agent, at any time after introduction due to intolerance or an adverse event.

In certain embodiments of any of the foregoing methods, the subject is intolerant to prior ESA treatment. In certain embodiments, the prior ESA-containing regimen, either as single agent or combination with other agent, at any time after introduction has been discontinued in the subject due to intolerance or an adverse event.

In certain embodiments of any of the foregoing methods, the subject is intolerant to prior ESA treatment. In certain embodiments, the subject has a low chance to respond to ESA treatments due to a high endogenous serum erythropoietin (EPO) level. In certain embodiments of any of the foregoing methods, the subject has not been previously treated with ESAs and has a serum EPO level>200 IU/L.

In certain embodiments, a subject treated in accordance with the methods provided herein has undergone prior treatment with one or more ESAs or is currently undergoing treatment with one or more ESAs. In certain embodiments, a subject treated in accordance with the methods provided herein does not respond to treatment with one or more ESAs. In certain embodiments, a subject treated in accordance with the methods provided herein is refractory to treatment with one or more ESAs. In certain embodiments, a subject treated in accordance with the methods provided herein becomes refractory to treatment with one or more ESAs. In certain embodiments, a subject treated in accordance with the methods provided herein is refractory to prior ESA treatment. In certain embodiments, a subject who is refractory to prior ESA treatment has documented non-response or is no longer responsive to prior ESA-containing regimen, either as single agent or combination with other agents (e.g., with G-CSF); the ESA regimen must have been either (a) recombinant human erythropoietin of greater than 40,000 IU/week for at least 8 doses or equivalent, or (b) darbepoetin alpha of greater than 500 ug once every three weeks for at least 4 doses or equivalent. In certain embodiments, a subject treated in accordance with the methods provided herein is intolerant to prior ESA-treatment. In certain embodiments, a subject who is intolerant to prior ESA-treatment has documented discontinuation of prior ESA-containing regimen, either as single agent or combination (e.g., with G-CSF), at any time after introduction due to intolerance or an adverse event. In certain embodiments, a subject treated in accordance with the methods provided herein is ESA-ineligible. In certain embodiments, a subject who is ESA-ineligible has a low chance of response to ESA based on an endogenous serum erythropoietin level of greater than 200 IU/L for subjects not previously treated with ESAs.

In certain embodiments, the subject treated in accordance with the methods described herein has MDS. In certain embodiments, the subject treated in accordance with the methods described herein has MDS and intact chromosome 5q. In certain embodiments, the subject treated in accordance with the methods provided herein has MDS, intact chromosome 5q, and does not have documented treatment failure with lenalidomide. In certain embodiments, the subject treated in accordance with the methods provided herein has MDS, intact chromosome 5q, and documented treatment failure with lenalidomide. In certain embodiments, the subject treated in accordance with the methods described herein has MDS with chromosome 5q deletion. MDS with chromosome 5q deletion comprises a deletion of the long arm of chromosome 5 and is characterized by, inter alia, macrocytic anemia with oval macrocytes, normal to slightly reduced white blood cell counts, normal to elevated platelet counts, and less than 5% blasts in the bone marrow and blood. In certain embodiments, the subject treated in accordance with the methods provided herein has MDS with chromosome 5q deletion and does not have documented treatment failure with lenalidomide. In certain embodiments, the subject treated in accordance with the methods provided herein has MDS with chromosome 5q deletion and documented treatment failure with lenalidomide. In certain embodiments, treatment failure with lenalidomide comprises loss of response to lenalidomide, no response to lenalidomide after 4 months of treatment with lenalidomide, intolerance to treatment with lenalidomide, or cytopenia precluding treatment with lenalidomide.

In certain embodiments, a subject treated in accordance with the methods provided herein has an EPO serum concentration of greater than 500 IU/L. In certain embodiments, a subject treated in accordance with the methods provided herein has an EPO serum concentration between 200 and 500 IU/L. In certain embodiments, a subject treated in accordance with the methods provided herein has an EPO serum concentration between 100 and 200 IU/L. In certain embodiments, a subject treated in accordance with the methods provided herein has an EPO serum concentration less than 100 IU/L.

In certain embodiments, a subject treated in accordance with the methods provided herein has a renal creatinine clearance rate between 40-60 mL/min. In certain embodiments, a subject treated in accordance with the methods provided herein has a renal creatinine clearance rate greater than 60 mL/min.

In certain embodiments, a subject treated in accordance with the methods provided herein has a baseline platelet count less than 100×10⁹/L. In certain embodiments, a subject treated in accordance with the methods provided herein has a baseline platelet count between 100 to 400 ×10⁹ /L. In certain embodiments, a subject treated in accordance with the methods provided herein has a baseline platelet count greater than 400×10⁹ /L.

In certain embodiments, a subject treated in accordance with the methods provided herein has received initial diagnosis of MDS between 0 to 2 years prior to the administration of luspatercept or sotatercept. In certain embodiments, a subject treated in accordance with the methods provided herein has received initial diagnosis of MDS between 2 to 5 years prior to the administration of luspatercept or sotatercept. In certain embodiments, a subject treated in accordance with the methods provided herein has received initial diagnosis of MDS more than 5 years prior to the administration of luspatercept or sotatercept.

In certain embodiments, provided herein is a method of treating a human subject who has been, or who is diagnosed with anemia due to very low, low, or intermediate risk myelodysplastic syndromes (MDS), comprising administering to the subject a therapeutically effective dose of luspatercept or sotatercept, wherein (a) the subject has at least 15% of erythroblasts that are ring sideroblasts, and (b) male subjects.

In certain embodiments, provided herein is a method of treating a human subject who has been, or who is diagnosed with anemia due to very low, low, or intermediate risk myelodysplastic syndromes (MDS), comprising administering to the subject a therapeutically effective dose of luspatercept or sotatercept, wherein (a) the subject has at least 15% of erythroblasts that are ring sideroblasts, and (b) subjects who have received initial diagnosis of MDS between 2 to 5 years prior to the administration of luspatercept or sotatercept.

In certain embodiments, provided herein is a method of treating a human subject who has been, or who is diagnosed with anemia due to very low, low, or intermediate risk myelodysplastic syndromes (MDS), comprising administering to the subject a therapeutically effective dose of luspatercept or sotatercept, wherein (a) the subject has at least 15% of erythroblasts that are ring sideroblasts, and (b) subjects having a baseline platelet count higher than 400×10⁹/L.

In certain embodiments, provided herein is a method of treating a human subject who has been, or who is diagnosed with anemia due to very low, low, or intermediate risk myelodysplastic syndromes (MDS), comprising administering to the subject a therapeutically effective dose of luspatercept or sotatercept, wherein (a) the subject has at least 15% of erythroblasts that are ring sideroblasts, and (b) subjects having a baseline serum erythropoietin (EPO) level of between 100 to 200 IU/L.

In certain embodiments, provided herein is a method of treating a human subject who has been, or who is diagnosed with anemia due to very low, low, or intermediate risk myelodysplastic syndromes (MDS), comprising administering to the subject a therapeutically effective dose of luspatercept or sotatercept, wherein (a) the subject has at least 15% of erythroblasts that are ring sideroblasts, and (b) subjects who have received 4 to 6 units of RBC transfusions during the 8-weeks period prior to the administration of luspatercept or sotatercept.

In certain embodiments, provided herein is a method of treating a human subject who has been, or who is diagnosed with anemia due to very low, low, or intermediate risk myelodysplastic syndromes (MDS), comprising administering to the subject a therapeutically effective dose of luspatercept or sotatercept, wherein (a) the subject has one or more mutations in SF3B1 gene, (b) the subject has at least 5% of erythroblasts that are ring sideroblasts, and (c) male subjects.

In certain embodiments, provided herein is a method of treating a human subject who has been, or who is diagnosed with anemia due to very low, low, or intermediate risk myelodysplastic syndromes (MDS), comprising administering to the subject a therapeutically effective dose of luspatercept or sotatercept, wherein (a) the subject has one or more mutations in SF3B1 gene, (b) the subject has at least 5% of erythroblasts that are ring sideroblasts, and (c) subjects who have received initial diagnosis of MDS between 2 to 5 years prior to the administration of luspatercept or sotatercept.

In certain embodiments, provided herein is a method of treating a human subject who has been, or who is diagnosed with anemia due to very low, low, or intermediate risk myelodysplastic syndromes (MDS), comprising administering to the subject a therapeutically effective dose of luspatercept or sotatercept, wherein (a) the subject has one or more mutations in SF3B1 gene, (b) the subject has at least 5% of erythroblasts that are ring sideroblasts, and (c) subjects having a baseline platelet count higher than 400×10⁹/L.

In certain embodiments, provided herein is a method of treating a human subject who has been, or who is diagnosed with anemia due to very low, low, or intermediate risk myelodysplastic syndromes (MDS), comprising administering to the subject a therapeutically effective dose of luspatercept or sotatercept, wherein (a) the subject has one or more mutations in SF3B1 gene, (b) the subject has at least 5% of erythroblasts that are ring sideroblasts, and (c) subjects having a baseline serum EPO level of between 100 to 200 IU/L.

In certain embodiments, provided herein is a method of treating a human subject who has been, or who is diagnosed with anemia due to very low, low, or intermediate risk myelodysplastic syndromes (MDS), comprising administering to the subject a therapeutically effective dose of luspatercept or sotatercept, wherein (a) the subject has one or more mutations in SF3B1 gene, (b) the subject has at least 5% of erythroblasts that are ring sideroblasts, and (c) subjects who have received 4 to 6 units of RBC transfusions during the 8-weeks period prior to the administration of luspatercept or sotatercept.

### 6. EXAMPLES

### 6.1 Example 1

### A Phase 3, Double-Blind, Randomized, Placebo-Controlled, Multicenter Study to Evaluate the Safety and Efficacy of Luspatercept (a Polypeptide Comprising an Amino Acid Sequence of SEQ ID NO: 1) for the Treatment of Anemia Due to IPSS-R Very Low, Low, or Intermediate Risk Myelodysplastic Syndromes in Subjects with Ring Sideroblasts Who Require Red Blood Cell Transfusions

### 6.1.1 Introduction

This example presents a Phase 3, double-blind, randomized, placebo-controlled, multicenter study to evaluate the safety and efficacy of luspatercept for the treatment of anemia due to IPSS-R very low, low, or intermediate risk myelodysplastic syndromes in subjects with ring sideroblasts who require red blood cell transfusions.

### 6.1.2 Results

229 MDS subjects who passed screening were enrolled as the randomized intent-to-treat (ITT) population. 153 of the 229 MDS subjects were enrolled in the treatment group and received luspatercept which started at 1 mg/kg dose level and can be dose titrated up to a maximum of 1.75 mg/kg. 76 of the 229 MDS subjects were enrolled in the placebo-controlled group.

The primary endpoint response rate was calculated using the number of responders divided by all subjects in the intent-to-treat (ITT) population. The study met its primary endpoint with statistical significance with respect to proportion of subjects who were red blood cell transfusion-independent eight weeks or longer after treatment, in favor of the luspatercept group over the placebo group. The secondary objectives were (i) to evaluate luspatercept effect on RBC transfusion independence (RBC-TI)≥12 weeks; (ii) to evaluate the effect of luspatercept on increase in hemoglobin; (iii) to evaluate the effect of luspatercept on the duration of RBC-TI; (iv) to evaluate the effect of luspatercept on time to RBC-TI; and (v) to evaluate the effect of luspatercept on erythroid hematological improvement (HI-E). The study also met statistical significance for the secondary efficacy endpoints of RBC-TI≥12 weeks and mHI-E during Study periods weeks 1-24 and weeks 1-48. In addition, the study showed a nonsignificant trend towards improvement in median duration of overall RBC-TI (in patients who responded in the first 24 weeks).

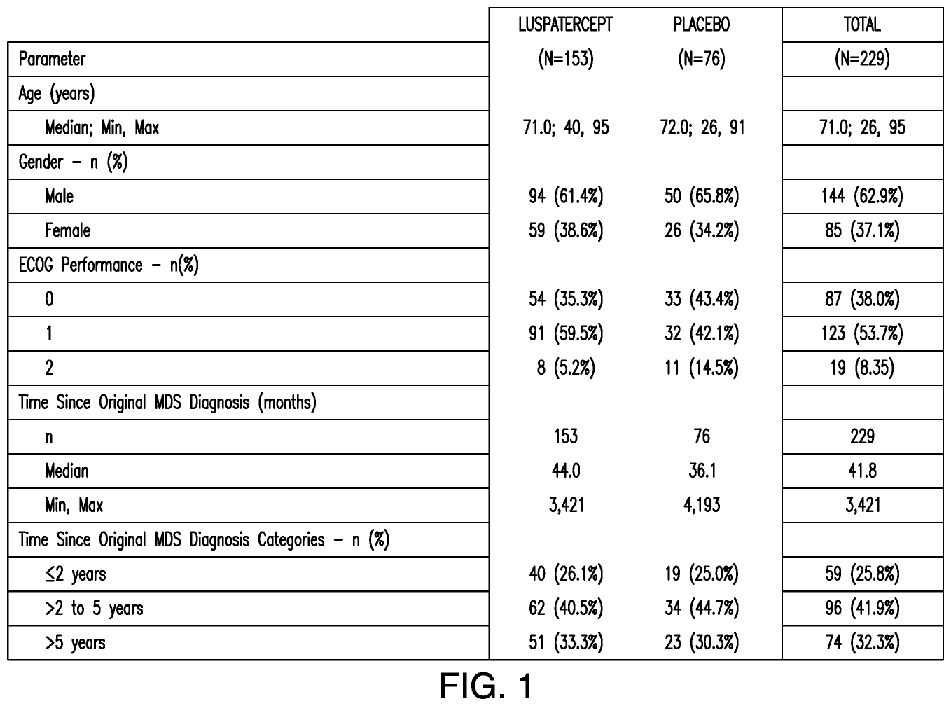

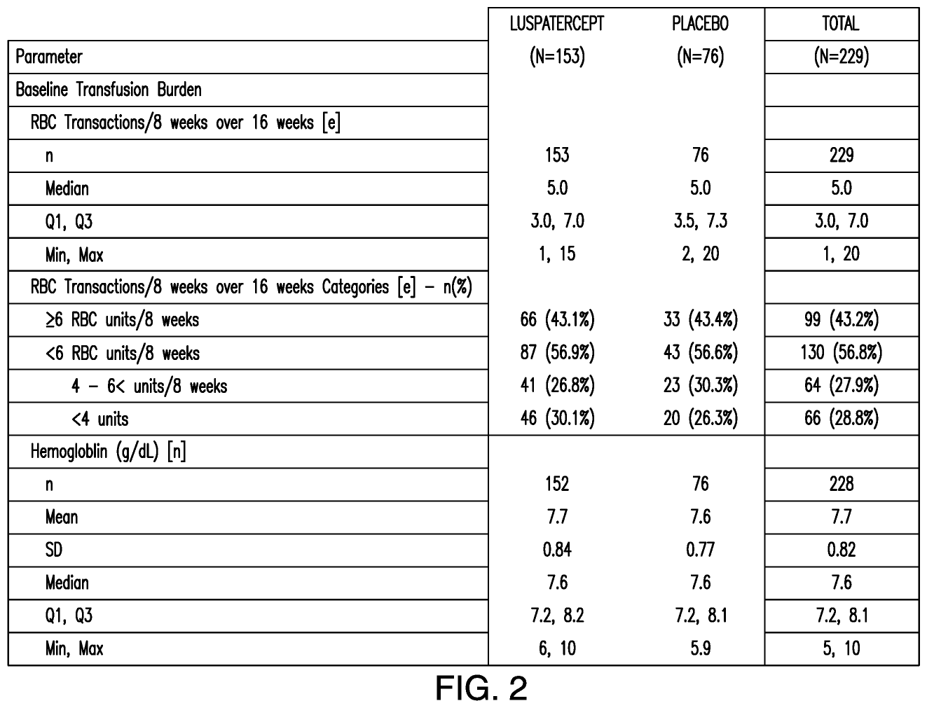

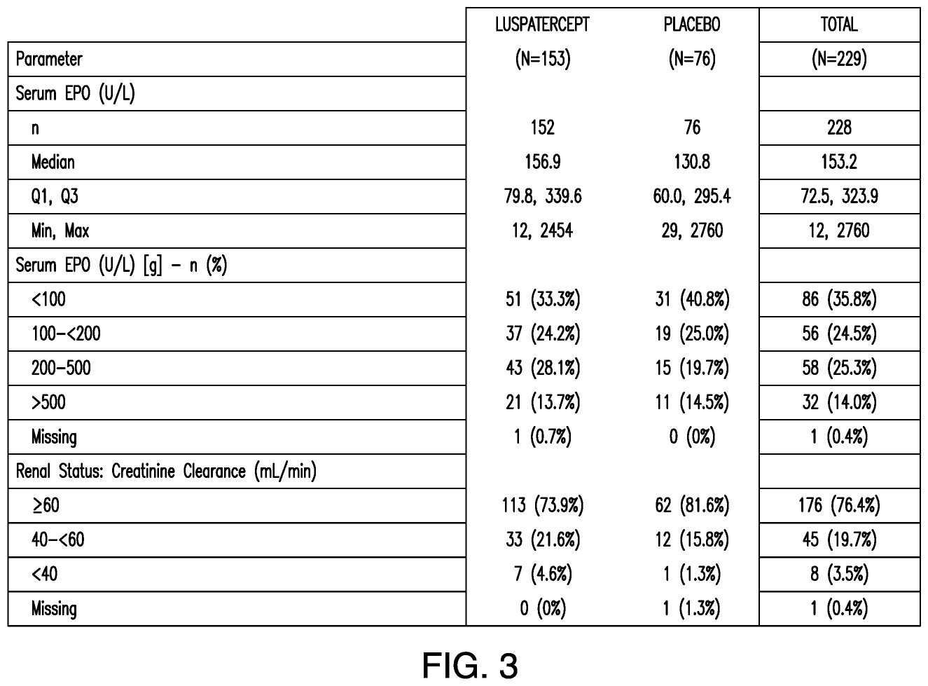

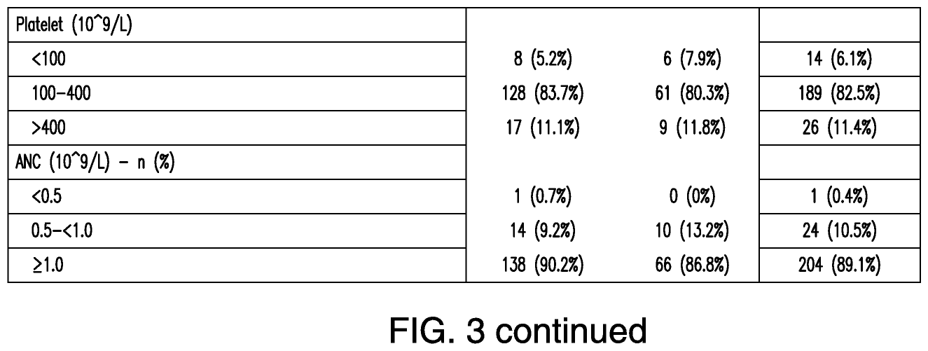

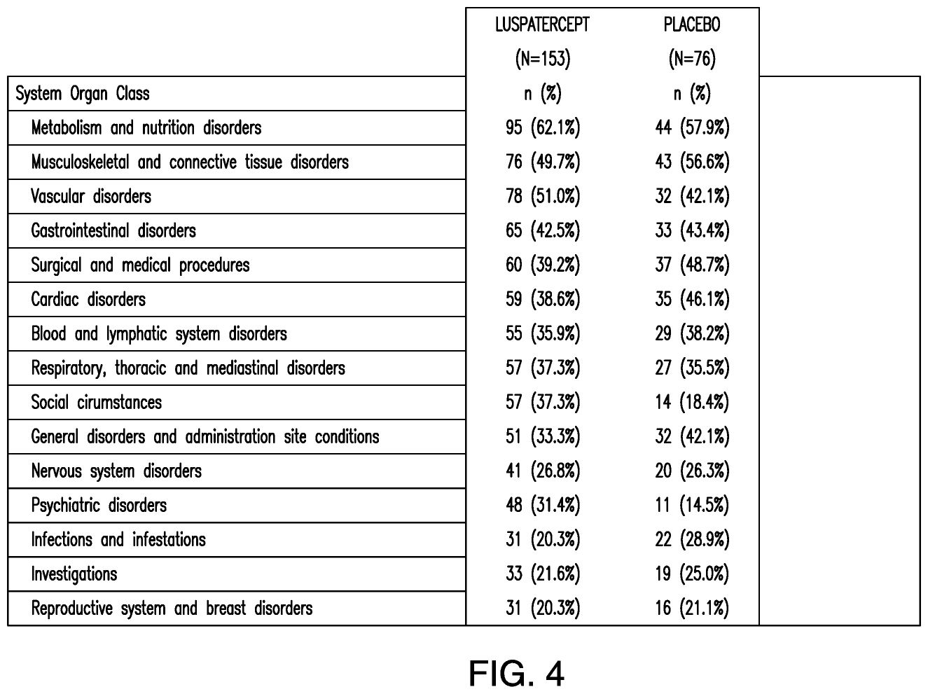

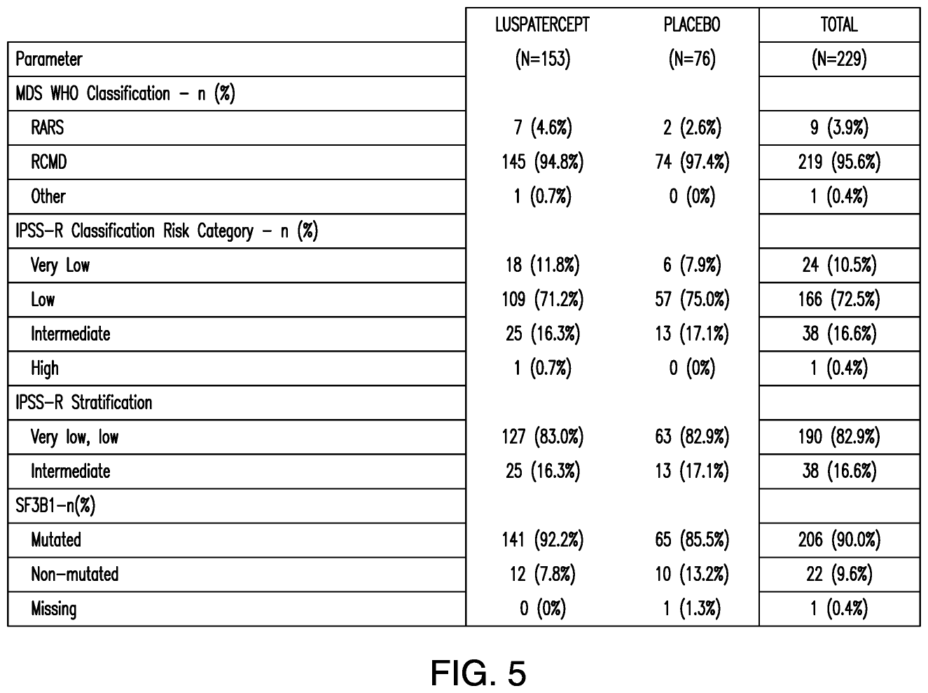

Subjects' age, weight, and baseline characteristics were summarized using descriptive statistics, while gender, race and other categorical variables were provided using frequency tabulations by dose cohort. See FIG. 1. Prior transfusion history was summarized. See FIG. 2. Medical history data was summarized using frequency tabulations by the Medical Dictionary for Regulatory Activities (MedDRA) system organ class and preferred term. See FIG. 3. Myelodysplastic syndrome (MDS) diagnoses as well as RBC transfusion dependence was summarized using frequency tabulations. See FIGS. 4 and 5.

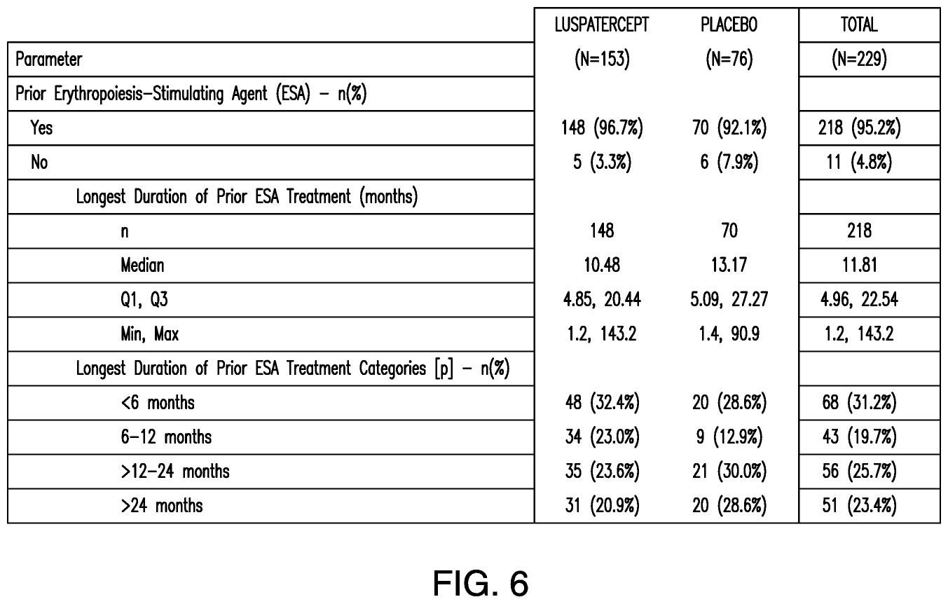

The efficacy analysis was performed on the ITT population. The efficacy endpoint are defined as: (i) Red Blood Cell Transfusion Independence (RBC-TI)≥8 weeks; (ii) Mean hemoglobin increase≥1.0 g/dL; (iii) Duration of RBC-TI; (iv) Time to RBC-TI; and (v) Erythroid response (HI-E). The primary efficacy endpoint result showed a statistically significant higher proportion of subjects in the treatment group achieved RBC-TI longer than 8 weeks during weeks 1-24, compared to placebo group. See FIG. 6. The key secondary endpoint analysis showed a statistically significant higher proportion of subjects in the treatment group achieved RBC-TI longer than 12 weeks.

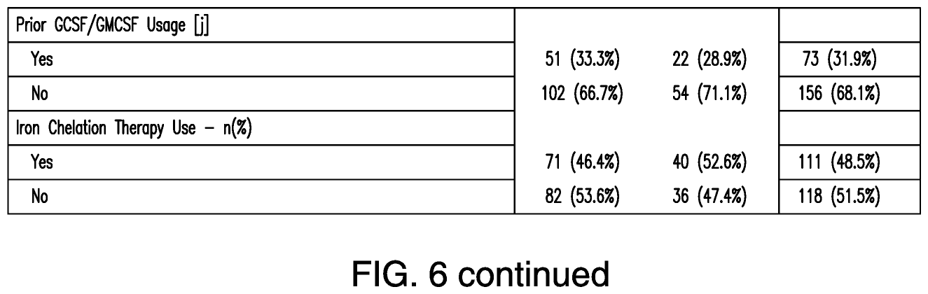

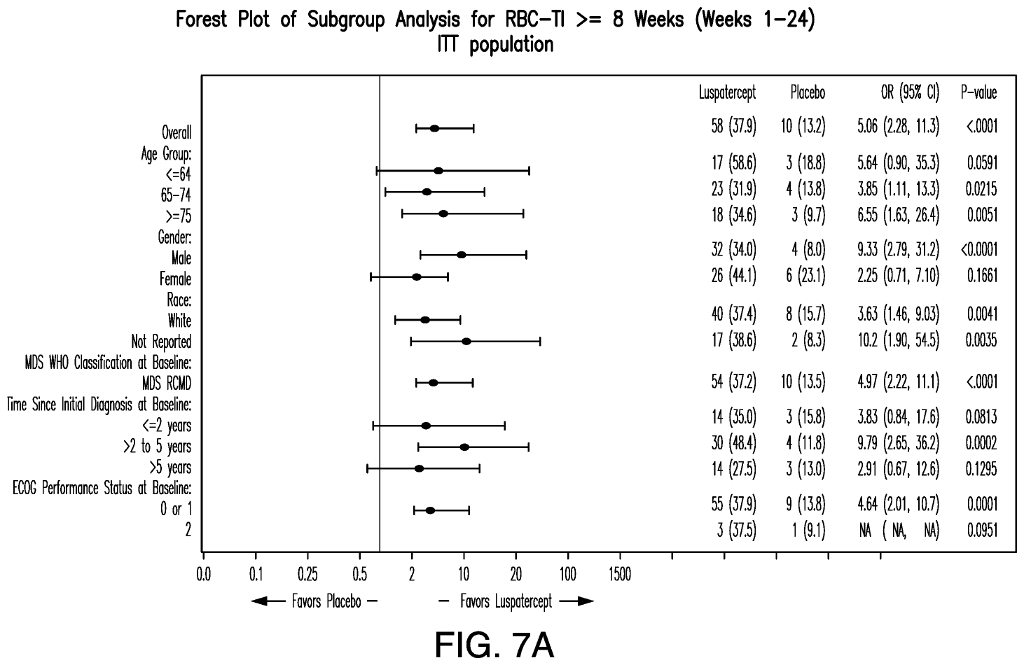

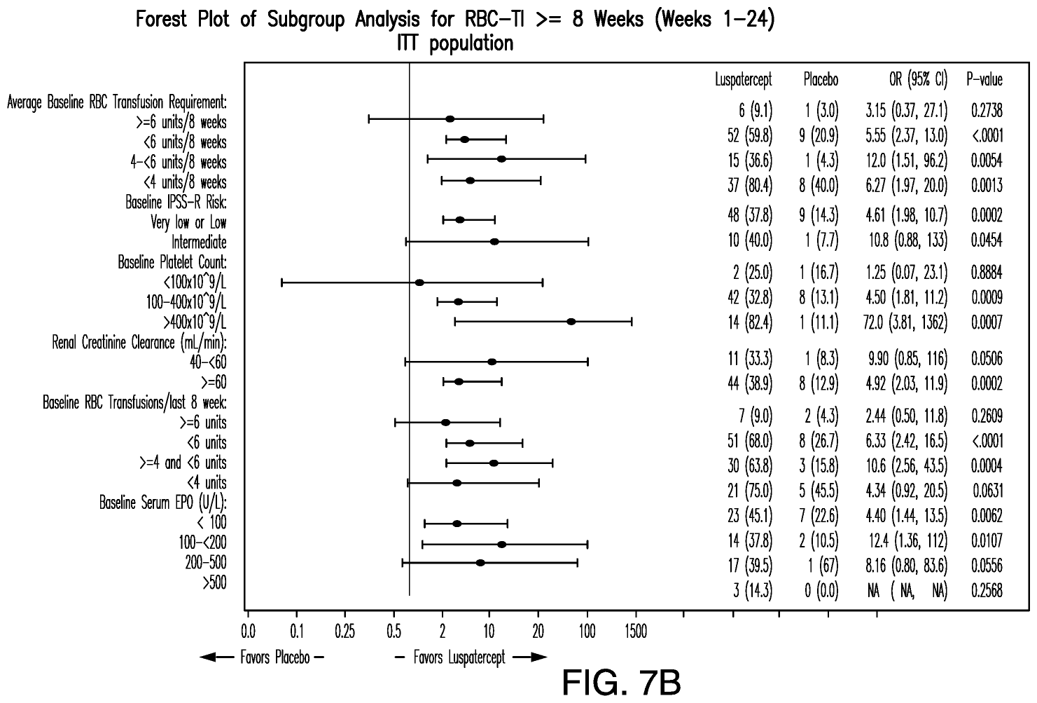

Forest plot of a subgroup analysis was performed for the ITT population with duration of RBC-TI longer than 8 weeks during weeks 1-24, while gender, race and other categorical variables were provided. The results were in favor of certain subgroups. See FIGS. 7A and 7B.

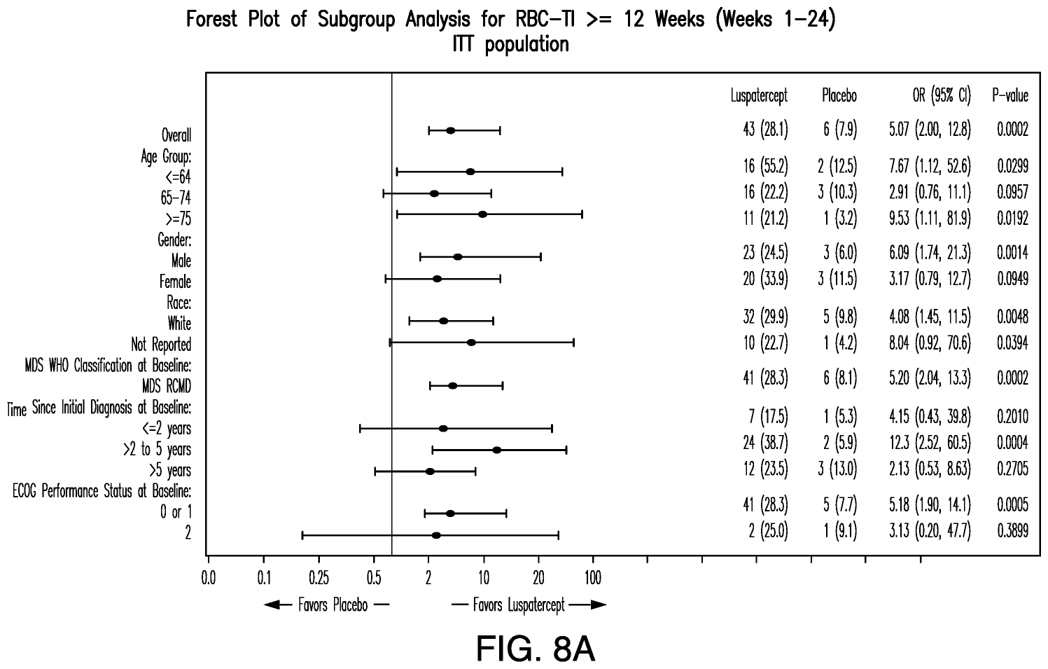

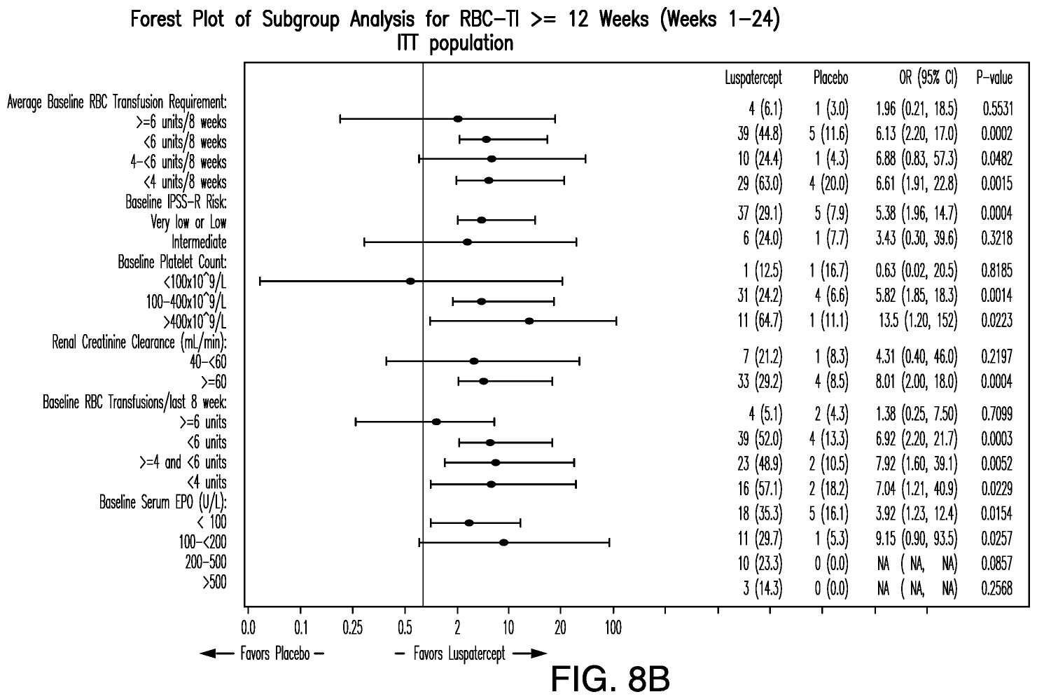

Similarly, forest plot of a subgroup analysis was performed for the ITT population with duration of RBC-TI longer than 12 weeks during weeks 1-24, while gender, race and other categorical variables were provided. The results were in favor of certain subgroups. See FIGS. 8A and 8B.

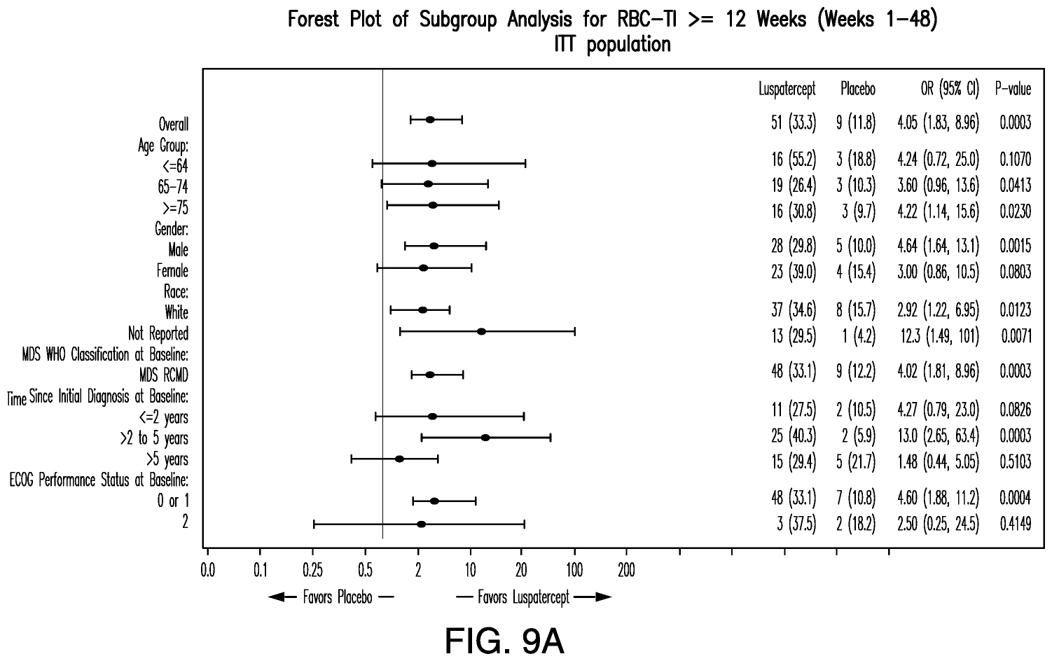

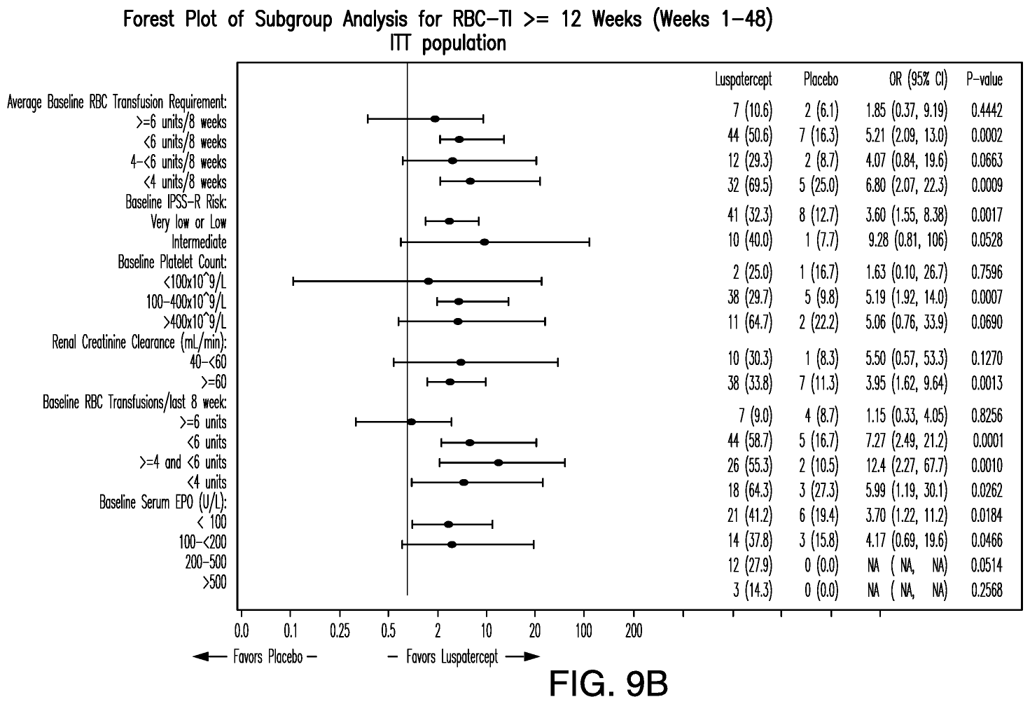

In addition, forest plot of a subgroup analysis was performed for the ITT population with duration of RBC-TI longer than 12 weeks during weeks 1-48, while gender, race and other categorical variables were provided. The results were in favor of certain subgroups. See FIGS. 9A and 9B.

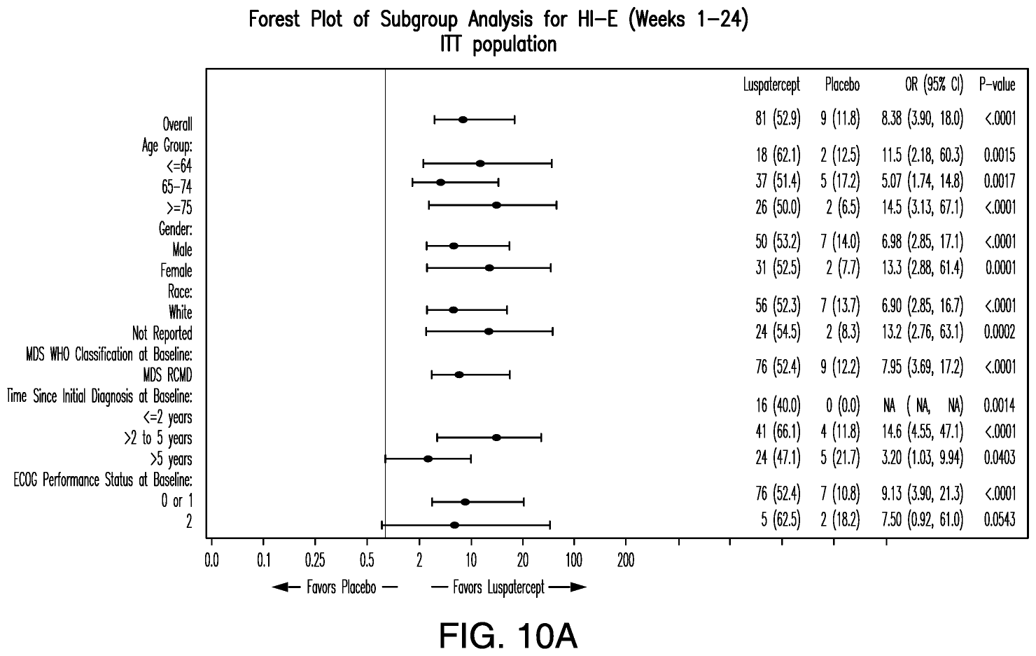

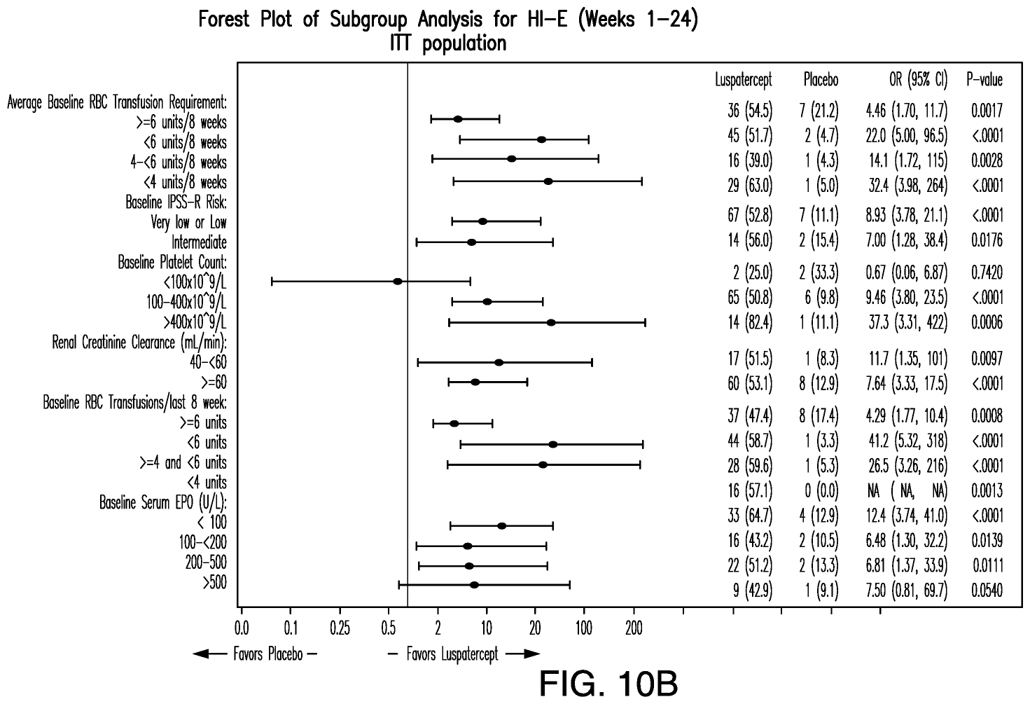

Furthermore, forest plot of a subgroup analysis was performed for modified erythroid response (mHI-E) during weeks 1-24, while gender, race and other categorical variables were provided. The results were in favor of certain subgroups. See FIGS. 10A and 10B.

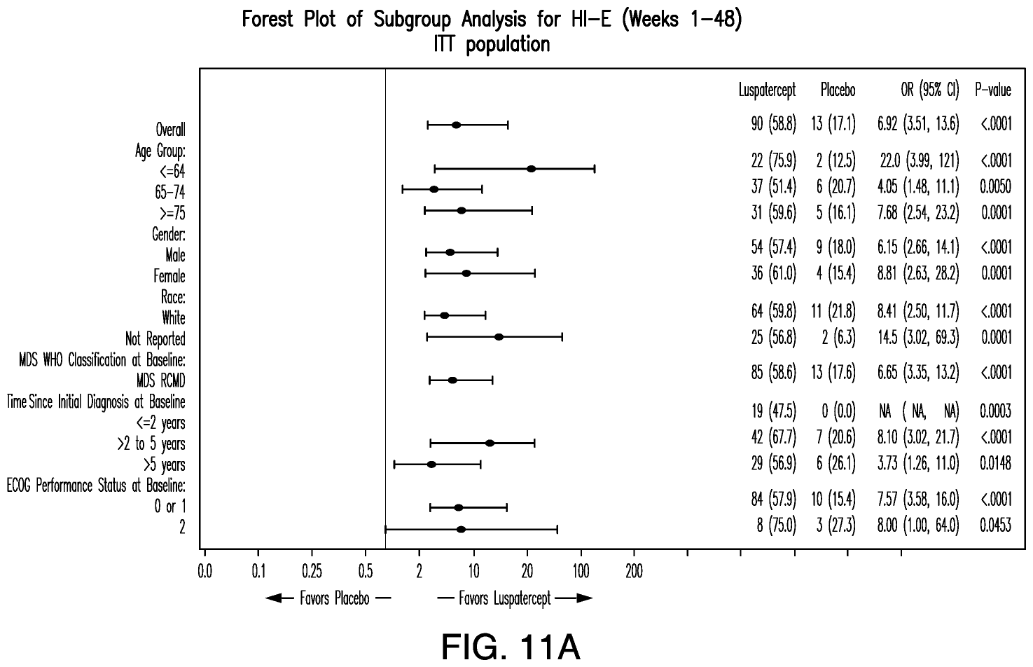

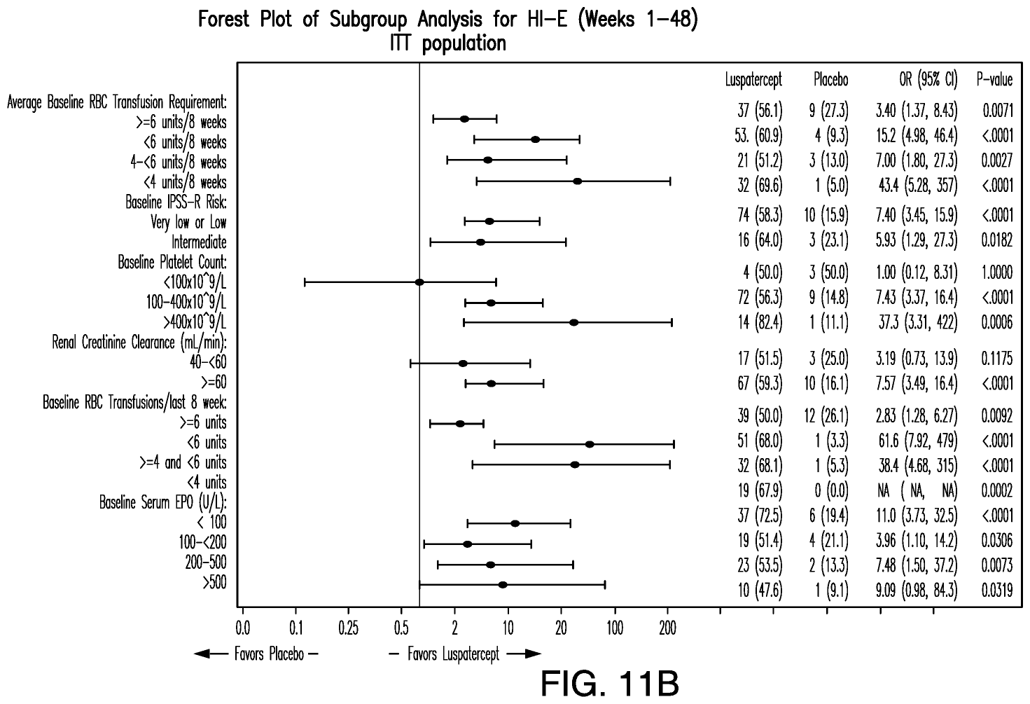

Similarly, forest plot of a subgroup analysis was performed for modified erythroid response (mHI-E) during weeks 1-48, while gender, race and other categorical variables were provided. The results were in favor of certain subgroups. See FIGS. 11A and 11B.

### 6.1.3 Study Design

Subjects received luspatercept subcutaneously on day 1 of each 21-day treatment cycle (initial dose of 1 mg/kg). Control subjects received placebo, subcutaneously, on day 1 of each 21-day treatment cycle. The treatment duration was a maximum of 24 months. The study was divided into: (i) a screening period, (ii) a treatment period, and (iii) a post treatment follow-up period.

(a) Screening Period

Upon giving written informed consent, the subject entered the screening period to determine eligibility. Subjects' identification (ID) number was allocated via Interactive Response Technology (IRT) system. Subject screening procedures were to take place within 28 days prior to Dose 1. During the screening period, the subject went through safety and other assessments to determine eligibility for the study. Re-screening was allowed, and a new subject ID number was assigned.

Local review of bone marrow aspirate smear and biopsy, peripheral blood smear, cytogenetics, were used to confirm MDS diagnosis and WHO classification and to determine the baseline IPSS-R risk classification (Greenberg et al., Blood, 2012; 120(12):2454-2465). RBC transfusion history must be available for at least the 16 weeks immediately preceding and including the date of enrollment and data should include the hemoglobin (Hgb) value for which the transfusion was administered (i.e., pre-transfusion Hgb value).

(b) Treatment Period

The subject entered the treatment period after the subject had fulfilled the required assessments in the screening period and had also fulfilled the eligibility criteria. After the subject enrollment via the IRT system, the first dose of luspatercept or placebo (Dose 1 Day 1) was administered within 3 days of enrollment and could be administered on the day of enrollment, provided that the eligibility criteria were met. Subjects received luspatercept or placebo on Day 1 of each 21-day treatment cycle. Best supportive care (BSC) was used in combination with study treatment when clinically indicated per investigator. Best supportive care included, but not limited to, treatment with transfusions, iron-chelating agents, antibiotic, antiviral and/or antifungal therapy, and nutritional support as needed. Best supportive care for this study excluded the use of ESAs.

(c) Post-Treatment Follow-Up Period

All adverse events (AEs) was recorded by the investigator from the time the subject signed informed consent until 42 days after the last dose of luspatercept or placebo as well as those serious adverse events (SAEs) made known to the investigator at any time thereafter that was suspected of being related to luspatercept or placebo. Transfusion data collection was continued until 42 days from the date of last dose of IP or the end of trial.

### 6.1.4 Study Objectives

TABLE 1
Study objectives of MDS subjects treated with luspatercept

Primary Objective
The primary objective of the study:
To evaluate RBC transfusion independence (RBC-TI) ≥ 8 weeks
(Time frame: week 1 through week 24)
Proportion of subjects who are red blood cell (RBC) transfusion free over
any consecutive 56-day period within week 1 through week 24
Secondary Objective(s)
The secondary objectives:
1. To evaluate RBC transfusion independence (RBC-TI) ≥ 12 weeks
(Time Frame: Up to approximately 48 weeks)
Proportion of subjects who are Red blood cell (RBC) transfusion free over
any consecutive 84-day period within week 1 through 24 and week 1
through 48
2. To evaluate RBC transfusion independence (RBC-TI) ≥ 8 weeks
(Time Frame: Up to approximately 48 weeks)
Proportion of subjects who are Red blood cell (RBC) transfusion free
over any consecutive 56-day period within week 1 through
3. To evaluate RBC transfusion independence (RBC-TI) over 16 weeks
(Time Frame: Up to approximately 48 weeks)
Mean change in total Red Blood Cell (RBC) units transfused over a fixed
16-week period within week 9 through 24 and week 33 through 48
4. Proportion of subjects achieving Modified hematologic improvement -
erythroid (mHI-E) per International Working Group (IWG) over any
consecutive 56 days
(Time Frame: Up to approximately 48 weeks)
Proportion of subjects achieving modified Hematological improvement-
erythroid (HI-E) over any consecutive 56-day period during the treatment
period
5. Mean hemoglobin increase ≥ 1.0 g/dL
(Time Frame: Up to approximately 48 weeks)
Proportion of subjects achieving hemoglobin (Hgb) increase from
baseline ≥ 1.0 g/dL over any consecutive 56-day period in absence of Red
blood cell (RBC) transfusions
6. Duration of Red Blood Cell Transfusion Independence (RBC-TI)
(Time Frame: Up to approximately 3.5 years)
Maximum duration of red blood cell (RBC) transfusion independence for
subjects who achieve RBC TI ≥ 8 weeks
7. Change in EORTC QLQ-C30 score
(Time Frame: Up to approximately 3.5 years)
Change in EORTC QLQ-C30 scores per scheduled visits
8. Hematologic improvement - neutrophils (HI-N) per International
Working Group (IWG)
(Time Frame: Up to approximately 48 weeks)
Proportion of subjects achieving HI-N over any consecutive 56-day period
9. Mean decrease in serum ferritin
(Time Frame: Up to approximately 48 weeks)
Change in serum ferritin over scheduled assessments
10. Mean decrease in iron chelation therapy (ICT) use
(Time Frame: Up to approximately 48 weeks)
Change in mean daily dose of ICT within week 9 through 24 and week
33 through 48
11. Time to red blood cell transfusion independence (RBC-TI)
(Time Frame: Up to approximately 48 weeks)
Time between randomization and the date onset of TI is first observed (ie,
Day 1 of 56 days without any RBC transfusions).
12. Number of subjects progressing to acute myeloid leukemia (AML)
(Time Frame: Up to approximately 3 years)
Assessments/measurements that were collected in different units of
measure will be aggregated and presented by standard units for the study.
13. Progression to acute myeloid leukemia (AML)
(Time Frame: Up to approximately 3 years)
Number and percentage of subjects progressing to AML and time to AML
progression from randomization through at least 3 years post last dose and
week 1 through 48
14. Overall survival (OS)
(Time Frame: Up to approximately 3.5 years)
Overall survival is defined as the time from date of randomization to death
due to any cause within randomization through at least 3 years post last
dose and week 1 through 48
15. Adverse Events (AEs)
(Time Frame: Up to approximately 3.5 years)
Type, frequency, severity of AEs and relationship of AEs to luspatercept/
placebo
16. Anti-drug antibodies (ADA)
(Time Frame: Up to approximately 3.5 years)
Frequency of anti-drug antibodies and effects on efficacy, or safety, or
PK from randomization through 1 year post first dose
17. Pharmacokinetics - AUC
(Time Frame: Up to approximately 1 year)
Area under the plasma concentration-time curve
18. Pharmacokinetics - Cmax
(Time Frame: Up to approximately 1 year)
Maximum observed concentration in plasma
19. Hematologic improvement- platelets (HI-P) per International Working
Group (IWG)
(Time Frame: Up to approximately 48 weeks)
Proportion of subjects achieving HI-P over any consecutive 56-day period
20. A population PK model and Exposure-Response relationship
(Time Frame: Up to approximately 3 years)
A population PK model that describes the PK exposure data of
luspatercept and associated variability from randomization through
1-year post dose.
Exposure-response relationship for the primary efficacy endpoint, AEs of
interest, and selected secondary endpoints from randomization through
1-year post first dose.

### 6.1.5 Inclusion Criteria

Subjects met the following criteria to be enrolled in the study: (i) subject was ≥18 years of age the time of signing the informed consent form (ICF); (ii) subject understood and voluntarily signed an ICF prior to any study-related assessments/procedures being conducted; (iii) documented local diagnosis of MDS according to WHO classification/French American British (FAB) classification that met IPSS-R classification (Greenberg et al., Blood, 2012; 120(12):2454-2465) of very low, low, or intermediate risk disease; (iv) subject must have had ring sideroblast ≥15% of erythroid precursors in bone marrow, or ≥5% (but <15%) if the subject had one or more mutations in SF3B1 gene; (v) subject must have had <5% blasts in bone marrow; (vi) subject must have had peripheral blood white blood cell (WBC) count <13,000/μL; (vii) subject must have required red blood cell RBC transfusions; (viii) subject must have had an Eastern Cooperative Oncology Group (ECOG) score of 0, 1, or 2; and (ix) subject was refractory or intolerant to, or ineligible for, prior ESA treatment. A subject who was refractory to prior ESA treatment could be a subject who have had a non-response or response that was no longer maintained to prior ESA-containing regimen, either as single agent or combination with other agent, at any time after introduction due to intolerance or an adverse event. A subject who was intolerant to prior ESA treatment had a prior ESA-containing regimen, either as single agent or combination with other agent, at any time after introduction discontinued due to intolerance or an adverse event. A subject who was ineligible for prior ESA treatment could be a subject who have had a low chance to respond to ESA treatments due to a high endogenous serum erythropoietin (EPO) level. In certain embodiments of any of the foregoing methods, the subject has not been previously treated with ESAs and has a serum EPO level >200 IU/L.

### 6.1.6 Exclusion Criteria

The presence of any of the following excluded a subject from enrollment: (i) prior therapy with disease modifying agents (e.g., immune-modulatory drug, hypomethylating agents, or immunosuppressive therapy) for underlying MDS disease; subjects who previously received hypomethylating agents (HMA) or lenalidomide could be enrolled at the investigator's discretion contingent on the subject's receiving no more than 2 doses of HMA or no more than 1 calendar week of treatment with lenalidomide; and the last dose must have been ≥5 weeks from the date of randomization.); (ii) prior treatment with either luspatercept or sotatercept; (iii) MDS associated with del 5q cytogenetic abnormality; (iv) secondary MDS, i.e., MDS that is known to have arisen as the result of chemical injury or treatment with chemotherapy and/or radiation for other diseases; (v) known clinically significant anemia due to iron, vitamin B₁₂, or folate deficiencies, or autoimmune or hereditary hemolytic anemia, or gastrointestinal bleeding; iron deficiency to be determined by serum ferritin≤15 μg/L and additional testing if clinically indicated (e.g., calculated transferrin saturation [iron/total iron binding capacity≤20%] or bone marrow aspirate stain for iron); (vi) prior allogeneic or autologous stem cell transplant; (vii) known history of diagnosis of acute myeloid leukemia (AML); (viii) use of any of the following within 5 weeks prior to randomization: anticancer cytotoxic chemotherapeutic agent or treatment, corticosteroid (except for subjects on a stable or decreasing dose for ≥1 week prior to randomization for medical conditions other than MDS), iron-chelating agents, except for subjects on a stable or decreasing dose for at least 8 weeks prior to randomization, other RBC hematopoietic growth factors (e.g., Interleukin-3), investigational drug or device, or approved therapy for investigational use; if the half-life of the previous investigational product is known, use within 5 times the half-life prior to randomization or within 5 weeks, whichever longer is excluded; (ix) uncontrolled hypertension, defined as repeated elevations of diastolic blood pressure (DBP) >100 mmHg despite adequate treatment; (x) absolute neutrophil count (ANC)<500/μL (0.5×10⁹/L); (xi) prior history of malignancies, other than MDS, unless the subject has been free of the disease (including completion of any active or adjuvant treatment for prior malignancy) for ≥5 years (subjects with the following history/concurrent conditions are allowed: basal or squamous cell carcinoma of the skin, carcinoma in situ of the cervix, and carcinoma in situ of the breast; and incidental histologic finding of prostate cancer (T1a or T1b using the tumor, nodes, metastasis [TNM] clinical staging system)); or (xii) major surgery within 8 weeks prior to randomization; subjects must have completely recovered from any previous surgery prior to randomization.

### 6.1.7 Treatment Dose, Administration, and Schedule

Luspatercept for injection was formulated as a sterile, preservative-free, lyophilized cake/powder. Luspatercept for injection was available in 2 strengths, and when reconstituted, consisted of 50 mg/mL of luspatercept in a 10 mM citrate buffer-based solution (10 mM citrate, pH=6.5, 9% sucrose, 0.02% polysorbate 80). The drug product was packaged in a 3 mL glass vial in the following strengths:

(i) 25 mg/vial: The 25 mg/vial presentation contained 37.5 mg of luspatercept. After reconstitution with 0.68 mL water for injection (WFI), each single-use vial delivers at least 0.5 mL of 50 mg/mL of luspatercept (25 mg); and

(ii) 75 mg/vial: The 75 mg/vial presentation contained 87.5 mg of luspatercept. After reconstitution with 1.6 mL WFI, each single-use vial delivers at least 1.5 mL of 50 mg/mL of luspatercept (75 mg).

Luspatercept or placebo was administered via subcutaneous (SC) injection to subjects by the study staff at the clinical site. Subjects had hemoglobin, blood pressure and weight assessed prior to each administration. Clinical site staff also confirmed if any transfusions were received by the subject (including any at outside local institutions in between study visits) prior to each administration via use of subject diary or other local procedure in place at the investigational site.

SC injections were given in the upper arm, thigh, and/or abdomen. Calculated doses requiring reconstituted volume greater than 1.2 mL were divided into separate similar volume injections across separate sites using the same anatomical location but on opposite sides of the body (example left thigh and right thigh). The maximum volume per SC injection was 1.2 mL.

The injection sites were rotated according to investigator's judgment, and the injections could be given in the following order as needed, for example: (i) right upper arm, (ii) left upper arm, (iii) right upper thigh, and (iv) left upper thigh.

The maximum total dose per administration was below 168 mg, which results in 3.36 mL maximum total volume after reconstitution.

The subject began treatment upon acknowledgement of eligibility by the sponsor. The subject was required to start the treatment within 28 days of signing the ICF. If screening assessments were performed within 72 hours of Cycle 1 Day 1 (C1D1), safety laboratory and physical examinations were not repeated at C1D1, with the exception of blood pressure measurement and hematology.

Subjects subcutaneously received luspatercept or placebo on Day 1 of each dosing cycle.

(a) Dose Modifications: Dose Titration, Dose Reduction, and Dose Delay

Starting as soon as Cycle 3 Day 1 and assessed by the investigator prior to every subsequent treatment cycle, subjects could have the dose level increased in a stepwise manner beyond the starting dose of 1.0 mg/kg to 1.33 mg/kg, and up to a maximum of 1.75 mg/kg, but the maximum total dose should not exceed 168 mg, if all the following criteria are met: (i) subject has ≥1 RBC transfusion event (for pre-transfusion Hgb of <9.0 g/dL) during the 2 most recent prior treatment cycles (˜6-weeks); (ii) the two most recent prior treatment cycles assessed must be at the same dose level; and (iii) subject must not have met protocol dose delay and/or reduction criteria in the two most recent treatment cycles (exception of dose delay required due to influence of RBC transfusions).

If all criteria above were met, the dose could be increased by 1 dose level. The dose level was titrated individually for each subject and did not exceed 1.75 mg/kg. Starting dose with dose increases and reductions are presented below for reference (Table 3).

TABLE 2
Starting dose level with dose reductions and dose titration for
MDS subjects

| 3^{rd} Dose | 2^{nd} Dose | 1^{st} Dose | | | |
|---|---|---|---|---|---|
| Reduction | Reduction | Reduction | Starting | 1^{st} Dose | 2^{nd} Dose |
| (~25% | (~25% | (~25% | Dose | Titration | Titration |
| reduction) | reduction) | reduction) | Level | Increase | Increase |
| 0.45 mg/kg | 0.6 mg/kg | 0.8 mg/kg | 1.0 mg/kg | 1.33 mg/kg | 1.75 mg/kg |

(b) Concomitant Medications and Procedures

Over the course of this study, additional medications was provided in some cases to manage aspects of the disease state of the subjects, including side effects from trial treatments or disease progression. Supportive care, including but not limited to anti-emetic medications, was administered at the discretion of the investigator.

Permitted concomitant medications and procedures included: (i) Granulocyte colony stimulating factors (i.e G-CSF, GM-CSF) were allowed only in cases of neutropenic fever or as clinically indicated per product label; (ii) concurrent corticosteroids used for medical conditions other than MDS was allowed provided subject was on a stable or decreasing dose for ≥1 week prior to randomization; and (iii) administration of attenuated vaccines (e.g., influenza vaccine) was allowed if clinically indicated, per investigator discretion; (iv) iron chelation therapy; subjects who were using iron-chelating therapies at time of randomization should be on a stable or decreasing dose for at least 8 weeks; concurrent treatment with iron chelation therapies during the Treatment Period was allowed at the discretion of the investigator and was recommended to be used per product label; and (vii) RBC transfusions; concurrent treatment for anemia with blood transfusions was allowed, at the discretion of the Investigator, for low hemoglobin levels, symptoms associated with anemia (e.g., hemodynamic or pulmonary compromise requiring treatment) or comorbidity.

Each subject has a “pre-transfusion hemoglobin threshold” for requiring transfusion during the study which was determined based on transfusion history. Baseline pre-transfusion hemoglobin threshold was the mean of all documented pre-transfusion hemoglobin values during the 16 weeks prior to Dose 1 Day 1. During treatment, if the pre-transfusion hemoglobin level was increased by ≥1 g/dL (at the time of a next anticipated transfusion event) compared to the pre-transfusion hemoglobin threshold for that subject, transfusion was delayed by a minimum of 7 days and/or the number of units transfused was reduced by 1 or more RBC units. Subjects could be transfused at the Investigator's discretion for symptoms related to anemia or other requirements (e.g., infection).

(c) Prohibited Concomitant Medications and Procedures

The following concomitant medications were specifically excluded during the course of the study: (i) cytotoxic, chemotherapeutic, targeted or investigational agents/therapies; (ii) azacitidine, decitabine or other hypomethylating agents; (iii) lenalidomide, thalidomide and other immunomodulating drugs (IMiDs); (iv) ESAs and other RBC hematopoietic growth factors (e.g., Interleukin-3); (v) G-CSF or GM-CSF, except in cases of neutropenic fever; (vi) hydroxyurea; (vii) androgens, unless to treat hypogonadism; (viii) oral retinoids (topical retinoids are permitted); (ix) arsenic trioxide; and (x) interferon (IFN).

### 7. DESCRIPTION OF THE SEQUENCES

TABLE 3
Sequence Information

| SEQ | | |
|---|---|---|
| ID | | |
| NO: | Description | Sequence |
| 1 | Processed ActRIIB-Fc | ETRECIYYNANWELERTNQSGLERCEGEQDKRLHCYA |
| | fusion protein with the | SWRNSSGTIELVKKGCWDDDFNCYDRQECVATEENPQ |
| | N-terminal 6 amino | VYFCCCEGNFCNERFTHLPEAGGPEVTYEPPPTGGGTH |
| | acids of the EC | TCPPCPAPELLGGPSVFLFPPKPKDTLMISRTPEVTCVV |
| | domain deleted and | VDVSHEDPEVKFNWYVDGVEVHNAKTKPREEQYNST |
| | the C-terminal 3 | YRVVSVLTVLHQDWLNGKEYKCKVSNKALPAPIEKTIS |
| | amino acids of the EC | KAKGQPREPQVYTLPPSREEMTKNQVSLTCLVKGFYPS |
| | domain deleted and | DIAVEWESNGQPENNYKTTPPVLDSDGSFFLYSKLTVD |
| | with an L79D | KSRWQQGNVFSCSVMHEALHNHYTQKSLSLSPGK |
| | mutation | |
| | (Luspatercept) | |
| 2 | Extracellular domain | ILGRSETQECLFFNANWEKDRTNQTGVEPCYGDKDKR |
| | of human ActRIIA | RHCFATWKNISGSIEIVKQGCWLDDINCYDRTDCVEKK |
| | fused to a human Fc | DSPEVYFCCCEGNMCNEKFSYFPEMEVTQPTSNPVTPK |
| | domain (Sotatercept) | PPTGGGTHTCPPCPAPELLGGPSVFLFPPKPKDTLMISRT |
| | | PEVTCVVVDVSHEDPEVKFNWYVDGVEVHNAKTKPR |
| | | EEQYNSTYRVVSVLTVLHQDWLNGKEYKCKVSNKAL |
| | | PVPIEKTISKAKGQPREPQVYTLPPSREEMTKNQVSLTC |
| | | LVKGFYPSDIAVEWESNGQPENNYKTTPPVLDSDGSFF |
| | | LYSKLTVDKSRWQQGNVFSCSVMHEALHNHYTQKSLS |
| | | LSPGKILGRSETQECLFFNANWEKDRTNQTGVEPCYGD |
| | | KDKRRHCFATWKNISGSIEIVKQGCWLDDINCYDRTDC |
| | | VEKKDSPEVYFCCCEGNMCNEKFSYFPEMEVTQPTSNP |
| | | VTPKPPTGGGTHTCPPCPAPELLGGPSVFLFPPKPKDTL |
| | | MISRTPEVTCVVVDVSHEDPEVKFNWYVDGVEVHNA |
| | | KTKPREEQYNSTYRVVSVLTVLHQDWLNGKEYKCKV |
| | | SNKALPVPIEKTISKAKGQPREPQVYTLPPSREEMTKNQ |
| | | VSLTCLVKGFYPSDIAVEWESNGQPENNYKTTPPVLDS |
| | | DGSFFLYSKLTVDKSRWQQGNVFSCSVMHEALHNHYT |
| | | QKSLSLSPGK |
| 3 | Human ActRIIB | SGRGEAETRECIYYNANWELERTNQSGLERCEGEQDK |
| | soluble (extracellular), | RLHCYASWANSSGTIELVKKGCWLDDFNCYDRQECV |
| | processed polypeptide | ATEENPQVYFCCCEGNFCNERFTHLPEAGGPEVTYEPP |
| | sequence | PTAPT |
| 4 | Human ActRIIA | ILGRSETQECLFFNANWEKDRTNQTGVEPCYGDKDKR |
| | soluble (extracellular), | RHCFATWKNISGSIEIVKQGCWLDDINCYDRTDCVEKK |
| | processed polypeptide | DSPEVYFCCCEGNMCNEKFSYFPEMEVTQPTSNPVTPK |
| | sequence | PP |
| 5 | A fragment of human | ETRECIYYNANWELERTNQSGLERCEGEQDKRLHCYA |
| | ActRIIB soluble | SWANSSGTIELVKKGCWLDDFNCYDRQECVATEENPQ |
| | (extracellular), | VYFCCCEGNFCNERFTHLPEAGGPEVTYEPPPT |
| | processed polypeptide | |
| | sequence (amino acids | |
| | 7-113 of SEQ ID NO: | |
| | 3) | |

### 8. EQUIVALENTS

Although the invention is described in detail with reference to specific embodiments thereof, it will be understood that variations which are functionally equivalent are within the scope of this invention. Indeed, various modifications of the invention in addition to those shown and described herein will become apparent to those skilled in the art from the foregoing description and accompanying drawings. Such modifications are intended to fall within the scope of the appended claims. Those skilled in the art will recognize, or be able to ascertain using no more than routine experimentation, many equivalents to the specific embodiments of the invention described herein. Such equivalents are intended to be encompassed by the following claims.

All publications, patents and patent applications mentioned in this specification are herein incorporated by reference into the specification to the same extent as if each individual publication, patent or patent application was specifically and individually indicated to be incorporated herein by reference in their entireties.

What is claimed:

1. A method of treating a human subject who has been, or who is diagnosed with, anemia due to very low, low, or intermediate risk myelodysplastic syndromes (MDS), comprising administering to the subject a therapeutically effective dose of luspatercept or sotatercept, wherein (a) the subject has at least 15% of erythroblasts that are ring sideroblasts, and (b) the subject falls into one of the following groups:
(i) male subjects;
(ii) subjects who have received initial diagnosis of MDS between 2 to 5 years prior to the administration of luspatercept or sotatercept;
(iii) subjects having a baseline platelet count higher than 400×10⁹/L;
(iv) subjects having a baseline serum erythropoietin (EPO) level of between 100 to 200 IU/L; and
(v) subjects who have received 4 to 6 units of RBC transfusions during the 8-weeks period prior to the administration of luspatercept or sotatercept.

2. A method of treating a human subject who has been, or who is diagnosed with, anemia due to very low, low, or intermediate risk myelodysplastic syndromes (MDS), comprising administering to the subject a therapeutically effective dose of luspatercept or sotatercept, wherein (a) the subject has one or more mutations in SF3B1 gene, (b) the subject has at least 5% of erythroblasts that are ring sideroblasts, and (c) the subject falls into one of the following groups:
(i) male subjects;
(ii) subjects who have received initial diagnosis of MDS between 2 to 5 years prior to the administration of luspatercept or sotatercept;
(iii) subjects having a baseline platelet count higher than 400×10⁹ /L;
(iv) subjects having a baseline serum EPO level of between 100 to 200 IU/L; and
(v) subjects who have received 4 to 6 units of RBC transfusions during the 8-weeks period prior to the administration of luspatercept or sotatercept.

3. The method of claim 1 or 2, wherein the very low, low, or intermediate risk MDS is categorized using International Prognostic Scoring System-Revised (IPSS-R).

4. The method of claim 1 or 2, wherein the subject is a subject has less than 5 percent of blasts in bone marrow.

5. The method of claim 1 or 2, wherein the subject is a subject requiring RBC transfusion.

6. The method of claim 1 or 2, wherein the method is a method to achieve (i) a long-term reduction in a percentage of erythroblasts in the subject that are ring sideroblasts as compared to an initial percentage of erythroblasts in the subject that are ring sideroblasts; and/or (ii) a long-term increase in hemoglobin level in the subject as compared to the hemoglobin level in the subject a period of time prior to administering to the subject an initial dose of the ActRII signaling inhibitor.

7. The method of claim 6, wherein the long-term is a duration of 8 weeks or more.

8. The method of claim 1 or 2, wherein the method is a method to achieve (i) a duration for red blood cell transfusion independence (RBC-TI) greater than or equal to 8 weeks in the subject after administration of luspatercept or sotatercept; (b) a duration for RBC-TI greater than or equal to 12 weeks in the subject after administration of luspatercept or sotatercept ring; or (c) a modified erythroid response (mHI-E) occurred in the subject after administration of luspatercept or sotatercept.

9. The method of claim 8, wherein the mHI-E is a mean hemoglobin increase of greater than or equal to 1.5 g/dL over 8 weeks, or reduction of 4 or more units of red blood cells transfused over 8 weeks, after said administering.

10. The method of claim 1, wherein the percentage of erythroblasts in the subject are ring sideroblasts prior to the administration of luspatercept or sotatercept is at least 15%, 16%, 17%, 18%, 19%, or at least 20%.

11. The method of claim 2, wherein the percentage of erythroblasts in the subject are ring sideroblasts prior to the administration of luspatercept or sotatercept is at least 5%, 6%, 7%, 8%, 9%, 10%, 11%, 12%, 13%, 14%, or at least 15%.

12. The method of claim 1 or 2, wherein the pharmaceutically effective dose of luspatercept or sotatercept is between 0.5 mg/kg and 1.75 mg/kg.

13. The method of claim 1 or 2, wherein the dose is about 0.45 mg/kg, 0.50 mg/kg, 0.60 mg/kg, 0.70 mg/kg, 0.80 mg/kg, 0.90 mg/kg, 1.00 mg/kg, 1.05 mg/kg, 1.10 mg/kg, 1.15 mg/kg, 1.20 mg/kg, 1.25 mg/kg, 1.30 mg/kg, 1.33 mg/kg, 1.35 mg/kg, 1.40 mg/kg, 1.45 mg/kg, 1.50 mg/kg, 1.55 mg/kg, 1.60 mg/kg, 1.65 mg/kg, 1.70 mg/kg, or 1.75 mg/kg.

14. The method of claim 1 or 2, wherein luspatercept or sotatercept is administered subcutaneously.

15. The method of claim 1 or 2, wherein the subject is refractory to prior erythropoiesis-stimulating agents (ESA) treatment.

16. The method of claim 1 or 2, wherein the subject is intolerant to prior ESA treatment.

17. The method of claim 1 or 2, wherein the subject is ineligible to ESA treatment.

18. The method of any of claims 15-17, wherein the ESA treatment is a treatment using ESA-containing regimens.

19. The method 18, wherein the ESA-containing regimen contains granulocyte-colony stimulating factor (G-CSF).

20. A pharmaceutical formulation of luspatercept, comprising: (i) a sterile, preservative-free, lyophilized cake or powder form of luspatercept before reconstitution, and (ii) a reconstituted form of luspatercept with concentration of 50 mg/mL in a 10 mM citrate buffer-based solution (10 mM citrate, pH=6.5, 9% sucrose, 0.02% polysorbate 80)

21. A single dosage pharmaceutical formulation comprising (i) a sterile, preservative-free, lyophilized cake or powder form of luspatercept before reconstitution, (ii) a reconstituted form of luspatercept with a concentration of 50 mg/mL in a 10 mM citrate buffer-based solution (10 mM citrate, pH=6.5, 9% sucrose, 0.02% polysorbate 80), and (iii) a 3 mL glass vial package contained 37.5 mg of luspatercept which delivers at least 0.5 mL of 50 mg/mL of luspatercept (25 mg/vial) after reconstitution with 0.68 mL water for injection (WFI).

22. A single dosage pharmaceutical formulation comprising (i) a sterile, preservative-free, lyophilized cake or powder form of luspatercept before reconstitution, (ii) a reconstituted form of luspatercept with a concentration of 50 mg/mL in a 10 mM citrate buffer-based solution (10 mM citrate, pH=6.5, 9% sucrose, 0.02% polysorbate 80), and (iii) a 3 mL glass vial package contained 87.5 mg of luspatercept which delivers at least 1.5 mL of 50 mg/mL of luspatercept (75 mg/vial) after reconstitution with 1.6 mL WFI.

23. The method of any of claims 1-19, wherein luspatercept or sotatercept is used as a pharmaceutical formulation in any of claims 20-22.


SEQUENCE LISTING (5 sequences)

SEQ ID NO: 1 - protein, 335 aa, Artificial Sequence
  Processed ActRIIB-Fc fusion protein with the N-terminal 6 amino acids of the EC domain deleted and the C-terminal 3 amino acids of the EC domain deleted and with an L79D mutation (Luspatercept)
ETRECIYYNANWELERTNQSGLERCEGEQDKRLHCYASWRNSSGTIELVKKGCWDDDFNCYDRQECVATEENPQVYFCCCEGNFCNERFTHLPEAGGPEVTYEPPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 688 aa, Artificial Sequence
  Extracellular domain of human ActRIIA fused to a human Fc domain (Sotatercept)
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 116 aa, Artificial Sequence
  Human ActRIIB soluble (extracellular), processed polypeptide sequence
SGRGEAETRECIYYNANWELERTNQSGLERCEGEQDKRLHCYASWANSSGTIELVKKGCWLDDFNCYDRQECVATEENPQVYFCCCEGNFCNERFTHLPEAGGPEVTYEPPPTAPT

SEQ ID NO: 4 - protein, 115 aa, Artificial Sequence
  Human ActRIIA soluble (extracellular), processed polypeptide sequence
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPP

SEQ ID NO: 5 - protein, 107 aa, Artificial Sequence
  A fragment of human ActRIIB soluble (extracellular), processed polypeptide sequence (amino acids 7-113 of SEQ ID NO 3)
ETRECIYYNANWELERTNQSGLERCEGEQDKRLHCYASWANSSGTIELVKKGCWLDDFNCYDRQECVATEENPQVYFCCCEGNFCNERFTHLPEAGGPEVTYEPPPT